SEQUENCE LISTING (38 sequences)

SEQ ID NO: 1 - protein, 189 aa, Homo sapiens
EPHSLRYNLTVLSGDGSVQSGFLAEVHLDGQPFLRCDRQKCRAKPQGQWAEDVLGNKTWDRETRDLTGNGKDLRMTLAHIKDQKEVLQSSDLGHERQEFLEGRCHEDQDTLSRYACRLPAGTTAISRIQRSPEENSAPHGECHPQRGLRGQHHRDMQGFQLLSPEYHTDLASGWGIFEPRHPAVGGCPA

SEQ ID NO: 2 - protein, 265 aa, Homo sapiens
EPHSLRYNLTVLSGDGSVQSGFLAEVHLDGQPFLRCDRQKCRAKPQGQWAEDVLGNKTWDRETRDLTGNGKDLRMTLAHIKDQKEVLQSSDLGHERQEFLEGRCHEDQDTLSRYACRLPAGTTAISKIRRSPEENSAPHGECHPQRGLEGQHYRDMQGFWLLSLEYHTELASGWGKVLVLQSHWQTFHVSAVAAAAAAAAAIFVIIIFYVCCCKKKTSAAEGPELVSLQVLDQHPVGTSDHRDATQLGFQPLMSDLGSTGSTEGA

SEQ ID NO: 3 - protein, 272 aa, Homo sapiens
EPHSLRYNLTVLSGDGSVQSGFLAEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNVRNFLKEDAMKTKTHYHAMHADCLQELRRYLKSGVVLRRTGKVLVLQSHWQTFHVSAVAAAAAAAAAIFVIIIFYVCCCKKKTSAAEGPELVSLQVLDQHPVGTSDHRDATQLGFQPLMSDLGSTGSTEGA

SEQ ID NO: 4 - protein, 173 aa, Homo sapiens
EPHSLRYNLTVLSGDGSVQSGFLAEVHLDGQPFLRCDRQKCRAKPQGQWAEDVLGNKTWDRETRDLTGNGKDLRMTLAHIKDQKEVLQSSDLGHERQEFLEGRCHEDQDTLSRYACRLPAGTTAISKIRRSPEENRSAGASESLADIPCFCCCCCCCCCCCYFCYYYFLRLLL

SEQ ID NO: 5 - protein, 176 aa, Homo sapiens
EPHSLRYNLTVLSGDGSVQSGFLAEVHLDGQPFLRCDRQKCRAKPQGQWAEDVLGNKTWDRETRDLTGNGKDLRMTLAHIKDQKEGKVLVLQSHWQTFHVSAVAAAAAAAAAIFVIIIFYVCCCKKKTSAAEGPELVSLQVLDQHPVGTSDHRDATQLGFQPLMSDLGSTGSTEGA

SEQ ID NO: 6 - DNA, 945 bp, Homo sapiens
gagccccacagtcttcgttataacctcacggtgctgtccggggatggatctgtgcagtcagggtttctcgctgaggtacatctggatggtcagcccttcctgcgctgtgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaagtcctccagagctcagaccttggccatgaacgtcaggaatttcttgaaggaagatgccatgaagaccaagacacactatcacgctatgcatgcagactgcctgcaggaactacggcgatatctagaatccagcgtagtcctgaggagaacagtgccccccatggtgaatgtcacccgcagcgaggcctcagagggcaacatcaccgtgacatgcagggcttccagcttctatccccggaatatcatactgacctggcgtcaggatggggtatctttgagccacgacacccagcagtggggggatgtcctgcctgatgggaatggaacctaccagacctgggtggccaccaggatttgccgaggagaggagcagaggttcacctgctacatggaacacagcgggaatcacagcactcaccctgtgcctctgggaaagtgctgtgcttcagagtcattggcagacattccatgttctgctgtgctgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagctcttgggtccactggctccactgagggcgcctag

SEQ ID NO: 7 - DNA, 797 bp, Homo sapiens
gagccccacagtcttcgttataacctcacggtgctgtccggggatggatctgtgcagtcagggtttctcgctgaggtacatctggatggtcagcccttcctgcgctgtgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagtgccccccatggtgaatgtcacccgcagcgaggcctcagagggcaacattaccgtgacatgcagggcttctggcttctatccctggaatatcacactgagctggcgtcaggatgggggaaagtgctggtgcttcagagtcattggcagacattccatgtttctgctgttgctgctgctgctgctgctgctgctgctatttttgttattattattttctacgtctgttgttgtaagaagaaaacatcagctgcagagggtccagagctcgtgagcctgcagtcctggatcaacacccagttgggacgagtgaccacagggatgccacacagctcggatttcagcctctgatgtcagatcttgggtccactggctccactgagggcgcctag

SEQ ID NO: 8 - DNA, 819 bp, Homo sapiens
gagccccacagtcttcgttataacctcacggtgctgtccggggatggatctgtgcagtcagggtttcttgctgaggtacatctggatggtcagcccttcctgcgctatgacaggcagaaatgcagggcaaagccccagggacagtgggcagaagatgtcctgggaaataagacatgggacagagagaccagsgacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactgaggaatggacagtgccccagtcctccagagctcagaccttggccatgaacgtcaggaatttcttgaaggaagatgccatgaagaccaagacacactatcacgctatgcatgcagactgcctgcaggaactacggcgatatctagaatccagcgtagtcctgaggagaacagggaaagtgctggtgcttcagagtcattggcagacattccatgtttctgctgttgctgctgctgctgctgctgctgctgctatttttgttattattattttctacgtctgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctag

SEQ ID NO: 9 - DNA, 682 bp, Homo sapiens
gagccccacagtcttcgttataacctcacggtgctgtccggggatggatctgtgcagtcagggtttctcgctgaggtacatctggatggtcagcccttcctgcgctgtgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagggaaagtgctggtgcttcagagtcattggcagacattccatgtttctgctgttgctgctgctgctgctgctgctgctgctatttttgttattattattttctacgtctgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctag

SEQ ID NO: 10 - DNA, 531 bp, Homo sapiens
gagccccacagtcttcgttataacctcacggtgctgtcctgggatggatctgtgcagtcagggtttcttgctgaggtacatctggatggtcagcccttcctgcgctatgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaagggaaagtgctggtgcttcagagtcattggcagacattccatgtttctgctgttgctgctgctgctgctgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctag

SEQ ID NO: 11 - DNA, 1188 bp, Homo sapiens
atggggctgggcccggtcttcctgcttctggctggcatcttcccttttgcacctccgggagctgctgctgagccccacagtcttcgttataacctcacggtgctgtccggggatggatctgtgcagtcagggtttctcgctgaggtacatctggatggtcagcccttcctgcgctgtgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaagtcctccagagctcagaccttggccatgaacgtcaggaatttcttgaaggaagatgccatgaagaccaagacacactatcacgctatgcatgcagactgcctgcaggaactacggcgatatctagaatccagcgtagtcctgaggagaacagtgccccccatggtgaatgtcacccgcagcgaggcctcagagggcaacatcaccgtgacatgcagggcttccagcttctatccccggaatatcatactgacctggcgtcaggatggggtatctttgagccacgacacccagcagtggggggatgtcctgcctgatgggaatggaacctaccagacctgggtggccaccaggatttgccgaggagaggagcagaggttcacctgctacatggaacacagcgggaatcacagcactcaccctgtgcctctgggaaagtgctgtgcttcagagtcattggcagacattccatgttctgctgtgctgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagctcttgggtccactggctccactgagggcgcctagactctacagccaggcggctggaattgaattccctgcctggatctcacaagcactttccctcttggtgcctcagtttcctgacctatgaaacagagaaaataaaagcacttatttattgttgttggaggctgcaaaatgttagtagatatgaggcatttgcagctgtgccatatt

SEQ ID NO: 12 - DNA, 1040 bp, Homo sapiens
atggggctgggcccggtcttcctgcttctggctggcatcttcccttttgcacctccgggagctgctgctgagccccacagtcttcgttataacctcacggtgctgtccggggatggatctgtgcagtcagggtttctcgctgaggtacatctggatggtcagcccttcctgcgctgtgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagtgccccccatggtgaatgtcacccgcagcgaggcctcagagggcaacattaccgtgacatgcagggcttctggcttctatccctggaatatcacactgagctggcgtcaggatgggggaaagtgctggtgcttcagagtcattggcagacattccatgtttctgctgttgctgctgctgctgctgctgctgctgctatttttgttattattattttctacgtctgttgttgtaagaagaaaacatcagctgcagagggtccagagctcgtgagcctgcagtcctggatcaacacccagttgggacgagtgaccacagggatgccacacagctcggatttcagcctctgatgtcagatcttgggtccactggctccactgagggcgcctagactctacagccaggcggctggaattgaattccctgcctggatctcacaagcactttccctcttggtgcctcagtttcctgacctatgaaacagagaaaataaaagcacttatttattgttgttggaggctgcaaaatgttagtagatatgaggcatttgcagctgtgccatatt

SEQ ID NO: 13 - DNA, 1062 bp, Homo sapiens
atggggctgggcccggtcttcctgcttctggctggcatcttcccttttgcacctccgggagctgctgctgagccccacagtcttcgttataacctcacggtgctgtccggggatggatctgtgcagtcagggtttcttgctgaggtacatctggatggtcagcccttcctgcgctatgacaggcagaaatgcagggcaaagccccagggacagtgggcagaagatgtcctgggaaataagacatgggacagagagaccagsgacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactgaggaatggacagtgccccagtcctccagagctcagaccttggccatgaacgtcaggaatttcttgaaggaagatgccatgaagaccaagacacactatcacgctatgcatgcagactgcctgcaggaactacggcgatatctagaatccagcgtagtcctgaggagaacagggaaagtgctggtgcttcagagtcattggcagacattccatgtttctgctgttgctgctgctgctgctgctgctgctgctatttttgttattattattttctacgtctgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctagactctacagccaggcggctggaattgaattccctgcctggatctcacaagcactttccctcttggtgcctcagtttcctgacctatgaaacagagaaaataaaagcacttatttattgttgttggaggctgcaaaatgttagtagatatgaggcatttgcagctgtgccatatt

SEQ ID NO: 14 - DNA, 925 bp, Homo sapiens
atggggctgggcccggtcttcctgcttctggctggcatcttcccttttgcacctccgggagctgctgctgagccccacagtcttcgttataacctcacggtgctgtccggggatggatctgtgcagtcagggtttctcgctgaggtacatctggatggtcagcccttcctgcgctgtgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagggaaagtgctggtgcttcagagtcattggcagacattccatgtttctgctgttgctgctgctgctgctgctgctgctgctatttttgttattattattttctacgtctgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctagactctacagccaggcggctggaattgaattccctgcctggatctcacaagcactttccctcttggtgcctcagtttcctgacctatgaaacagagaaaataaaagcacttatttattgttgttggaggctgcaaaatgttagtagatatgaggcatttgcagctgtgccatatt

SEQ ID NO: 15 - DNA, 774 bp, Homo sapiens
atggggctgggcccggtcttcctgcttctggctggcatcttcccttttgcacctccgggagctgctgctgagccccacagtcttcgttataacctcacggtgctgtcctgggatggatctgtgcagtcagggtttcttgctgaggtacatctggatggtcagcccttcctgcgctatgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaagggaaagtgctggtgcttcagagtcattggcagacattccatgtttctgctgttgctgctgctgctgctgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctagactctacagccaggcggctggaattgaattccctgcctggatctcacaagcactttccctcttggtgcctcagtttcctgacctatgaaacagagaaaataaaagcacttatttattgttgttggaggctgcaaaatgttagtagatatgaggcatttgcagctgtgccatatt

SEQ ID NO: 16 - DNA, 21 bp, Artificial
  Synthetic oligonucleotide primer
ggacagtgggcagaagatgtc

SEQ ID NO: 17 - DNA, 22 bp, Artificial
  Synthetic oligonucleotide primer
gctctggaggacttctttctgg

SEQ ID NO: 18 - DNA, 23 bp, Artificial
  Synthetic oligonucleotide primer
gccatgaagaccaagacacacta

SEQ ID NO: 19 - DNA, 19 bp, Artificial
  Synthetic oligonucleotide primer
accagcactttcccccatc

SEQ ID NO: 20 - DNA, 23 bp, Artificial
  Synthetic oligonucleotide primer
gccatgaagaccaagacacacta

SEQ ID NO: 21 - DNA, 21 bp, Artificial
  Synthetic oligonucleotide primer
ccagcactttccctgttctcc

SEQ ID NO: 22 - DNA, 22 bp, Artificial
  Synthetic oligonucleotide primer
ccagaaagaagtcctccagagc

SEQ ID NO: 23 - DNA, 21 bp, Artificial
  Synthetic oligonucleotide primer
gcactttccctgttctcctca

SEQ ID NO: 24 - DNA, 19 bp, Artificial
  Synthetic oligonucleotide primer
tggatggtcagcccttcct

SEQ ID NO: 25 - DNA, 22 bp, Artificial
  Synthetic oligonucleotide primer
gcactttcccttctttctggtc

SEQ ID NO: 26 - DNA, 20 bp, Artificial
  Synthetic oligonucleotide primer
acgcgttgtctgtcctggaa

SEQ ID NO: 27 - DNA, 20 bp, Artificial
  Synthetic oligonucleotide primer
gaggtgcaaaagggaagatg

SEQ ID NO: 28 - DNA, 23 bp, Artificial
  Synthetic oligonucleotide primer
atttcctgccccaggaaggttgg

SEQ ID NO: 29 - DNA, 20 bp, Artificial
  Synthetic oligonucleotide primer
agacaggtccctgctctctg

SEQ ID NO: 30 - DNA, 23 bp, Artificial
  Synthetic oligonucleotide primer
aaatgccttcatccatagcacag

SEQ ID NO: 31 - DNA, 21 bp, Artificial
  Synthetic oligonucleotide primer
gacttgcaggtcaggggtccc

SEQ ID NO: 32 - DNA, 21 bp, Artificial
  Synthetic oligonucleotide primer
tgtccctaccctggcctgacc

SEQ ID NO: 33 - DNA, 21 bp, Artificial
  Synthetic oligonucleotide primer
cctttttttcagggaaagtgc

SEQ ID NO: 34 - DNA, 22 bp, Artificial
  Synthetic oligonucleotide primer
ccttaccatctccagaaactgc

SEQ ID NO: 35 - DNA, 21 bp, Artificial
  Synthetic oligonucleotide primer
gatgttgatggagtgatggga

SEQ ID NO: 36 - DNA, 24 bp, Artificial
  Synthetic oligonucleotide primer
atgttgatcaggatggtctcgatc

SEQ ID NO: 37 - DNA, 17 bp, Artificial
  Synthetic oligonucleotide primer
gtcggggccatggggct

SEQ ID NO: 38 - DNA, 21 bp, Artificial
  Synthetic oligonucleotide primer
tcataggtcaggaaactgagg

(19) United States
(12) Patent Application Publication
(10) Pub. No.: US 2016/0176943 A1
(43) Pub. Date: Jun. 23, 2016

(54) NOVEL ALTERNATIVE SPLICE TRANSCRIPTS FOR MHC CLASS I RELATED CHAIN ALPHA (MICA) AND USES THEREOF

(71) Applicants: INSERM (INSTITUT NATIONAL DE LA SANTÉ ET DE LA RECHERCHE MÉDICALE), Paris (FR); UNIVERSITÉ DE NANTES, Nantes (FR); CHU DE NANTES, Nantes (FR)

(72) Inventors: Beatrice CHARREAU, Nantes (FR); Nathalie GERARD, Nantes (FR); Pierre TONNERRE, Nantes (FR); Pierre-Jean GAVLOVSKY, Nantes (FR)

(73) Assignee: INSERM (INSITITUT NATIONAL DE LA SANTE ET DE LA RECHERCHE MEDICALE), Paris (FR)

(21) Appl. No.: 14/902,615

(22) PCT Filed: Jul. 4, 2014
(86) PCT No.: PCT/EP2014/064316

§ 371 Date: Jan. 4, 2016

(30) Foreign Application Priority Data
Jul. 5, 2013 (EP) 13305955.0

(51) Int. Cl. C07K 14/74; G01N 33/68; C12Q 1/68

(52) U.S. Cl. CPC C07K 14/70539; C12Q 1/6883; G01N 33/6893; C12Q 2600/156

(57) ABSTRACT

The present invention relates to novel alternative splice transcripts (AST) for MICA (MHC class I related chain alpha) encoding novel MICA protein isoforms and uses thereof. In particular, the present invention relates to an isolated polypeptide at least 80% of identity with a sequence selected from the group consisting of SEQ ID NO:1 (MICA-A), SEQ ID NO:2 (MICA-B1), SEQ ID NO:3 (MICA-B2); SEQ ID NO:4 (MICA-C) and SEQ ID NO: (MICA-D).

### FIELD OF THE INVENTION

The present invention relates to novel alternative splice transcripts (AST) for MICA (MHC class I related chain alpha) encoding novel MICA protein isoforms and uses thereof.

### BACKGROUND OF THE INVENTION

The classical HLA class I loci within the MHC (HLA-A, -B, -C) are characterized by their ubiquitous expression and their wide polymorphism {Mason, 1998 #39}. By contrast, the human MHC class I chain-related genes (MICA and MICB), located within the HLA class I region of chromosome 6, show a restricted cell and tissue distribution (Bahram et al., 1996). Moreover, their structure organization, expression and products differ considerably from classical HLA class I genes (Groh et al., 1996). MICA are constitutively expressed on the cell surface of freshly isolated gastric epithelium, ECs and fibroblasts, but are not present on CD4+ and CD8+ T cells or B cells unless activated (Groh et al., 1996) (Zwirner et al., 1998) (Zwirner et al., 1999).

Expression of MICA proteins is considerably deregulated or increased in transformed cells of various types, particularly in those of an epithelial origin suggesting a role for MICA in tumor's immune escape. Moreover, early evidence for heat and virus-induced upregulation of MIC protein cell-surface expression has led to the concept that MIC proteins are markers of stress in the epithelia (Bauer et al., 1999). The MIC genes are highly polymorphic (Stephens, 2001) (Bahram et al., 2005) and more than 94 alleles have been reported yet. It is likely that the polymorphic MICA molecule may be target for specific antibodies and T cells in solid organ grafts (Hankey et al., 2002) (Sumitran-Holgersson et al., 2002) (Collins et al., 2002). It was recently showed that MICA polymorphic variant can trigger selective MICA expression on ECs promoting alloimmune response in solid organ transplantation (Tonnerre P. et al. 2012, manuscript in revision). Nevertheless, the specific impact that MICA polymorphism can play on NKG2D-mediated immune responses is almost unknown. Functionally, MICA, unlike classical HLA class I molecules, do not bind β2-microglobulin (β2-m) and are independent of any transporter-associated protein (TAP) which exclude a role for MICA in peptide binding and antigen presentation (Groh et al., 1996). MICA is a ligand for the activating immunoreceptor NKG2D, a highly conserved C-type lectinlike membrane glycoprotein expressed on essentially all natural killer (NK) cells, as well as on γδ and αβ CD8(+) T cells, in humans and mice (for review see (Raulet, 2003) (Gonzalez et al., 2008; Ogasawara and Lanier, 2005)). NKG2D participates in both innate and adaptive immunity. Its physiological roles include facilitating surveillance against microbial and viral infections and cancer, whilst it is also involved in the pathogenesis of some autoimmune diseases (Type 1 diabetes (T1D), celiac disease (CD), rheumatoid arthritis (RA)) and in allograft rejection (Caillat-Zucman, 2006; Ogasawara and Lanier, 2005; Suarez-Alvarez et al., 2009b). Depending upon the situation, development of strategies to either block or to enhance the interactions between NKG2D and its ligands may have important implications for human health and disease (Mondelli, 2012).

The estimated number of genes that encode more than one protein as a result of alternative splicing of a pre-mRNA has steadily risen over time. Recent studies using high throughput sequencing indicate that 95-100% of human pre-mRNAs that contain sequence corresponding to more than one exon are processed to yield multiple mRNAs (Nilsen and Graveley, 2010). Although the functional relevance of mRNA isoforms is still unclear, studies provided numerous examples in which alternative splicing clearly gives rise to functionally distinct isoforms (Wang and Burge, 2008). Alternatively spliced isoforms are known to exist for HLA-A and B (Krangel, 1986), as well as HLA-G (Ishitani and Geraghty, 1992) and some MHC class I-related genes such as EPCR (Saposnik et al., 2008) and MR1(Riegert et al., 1998). In a previous study, Zou and Stasny reported on alternate transcripts for MICA and MICB in the colon carcinoma cell line HCT 116. In these cells, they found 2 cDNAs encoding a 1161-bp cDNA, representing full-length MICA or MICB, and a shorter variant of 873 bp. The sequences of the short cDNAs correspond to MICA or MICB alleles lacking exon 3. These putative additive transcripts were called MICA2 and MICB2 (Zou and Stastny, 2002). However, the relevance of these transcripts still remains unclear.

### SUMMARY OF THE INVENTION

The present invention relates to novel alternative splice transcripts (AST) for MICA (MHC class I related chain alpha) encoding novel MICA protein isoforms and uses thereof.

### DETAILED DESCRIPTION OF THE INVENTION

The inventors have now identified five novel alternative splice transcripts (AST) for MICA (MHC class I related chain alpha) in human endothelial cells (ECs). They demonstrated that alternative transcripts (AT) result from a point deletion (G) in the 5′ splice donor site of MICA intron 4 leading to exon3 and exon4 skipping and/or deletions. They found that this deletion in the 5′ splice donor site of MICA intron 4 is a typical feature of at least two MICA alleles (MICA *015 and *017) and no alternative transcripts were found associated with the most frequent MICA alleles (MICA*002, *004, *008, *009, *011 . . . ). A set of primer pairs was designed to selectively detect and quantify the five MICA alternate transcripts in cells by RTPCR. Using their dedicated PCR assays, the inventors were able to demonstrate the presence of AT in ECs from MICA*015 or*017 individuals but also in PBMC suggesting that these MICA isoforms are not restricted to a cell type (i.e. ECs) but instead are expressed in both hematopoietic and non hematopoietic cells. Importantly, in cells homozygous for the mutation and expressing MICA AT no mRNA for MICA WT was detected. Cloning and transfection of the five full cDNAs in COS-7 cells confirmed the expression of the 5 alternative transcripts at mRNA level. Transfectants were used to investigate MICA isoform expression by Western blotting, flow cytometry, immunohistology and confocal analysis. First, the inventors found that all alternate transcripts give rise to stable proteins in transfected cells. In contrast to the other 4 isoforms, lower protein expression was consistently detected for MICA-A isoform suggesting that this isoform was less stable than others. Isoforms MICA-B1, -B2, C and D were consistently detected by flow cytometry and intracellular immunofluorescence using anti-Flag (M2) antibodies. Results from confocal imaging support a membrane-bound expression for isoforms MICA-B1, -B2 and -D. Further characterization indicated that MICA-B1 and MICA-B2 are membrane-bound isoforms of 29.4 kDa and 30.4 kDa, respectively, recognized by usual anti-MICA antibodies (clone AM01 directed against α1 and α2 domains). The inventors also showed that anti-MICA antibodies in the sera of sensitized transplant recipients are able to bind to, at least, MICA-B2 suggesting that this isoform could play a role in an allogeneic response in organ transplantation. Functionally, partial or complete deletion of exons 3 and 4 in the AT induces the lack of α3 extracellular domain in all isoforms and α2 domain in the majority of isoforms (A, B1, C and D). Indeed, only MICA-B2 contains two extracellular domains (α1 and α2). For isoforms MICA-A, MICA-B1 and MICA-C, deletions cause change in ORF and generate a premature stop codon creating new AA sequences with partial sequence homology for the 3 isoforms. The inventors demonstrated that MICA-B2 is a novel ligand for the activating receptor NKG2D and a potential target for allospecific antibodies in transplanted patients. To conclude, the inventors demonstrated the occurrence of novel MICA gene alternative splicing associated with MICA gene polymorphic variants leading to expression of novel isoforms in vascular and hematopeitic cells and providing new ligands for the activating NKG2D immune receptor.

Accordingly an aspect of the invention relates to an isolated polypeptide having a sequence selected from the group consisting of SEQ ID NO:1 (MICA-A), SEQ ID NO:2 (MICA-B1), SEQ ID NO:3 (MICA-B2); SEQ ID NO:4 (MICA-C) and SEQ ID NO:5 (MICA-D).

(MICA-A)
AA1-AA189
SEQ ID NO 1
EPHSLRYNLTVLSGDGSVQSGFLAEVHLDGQPFLRCDRQKCRAK
PQGQWAEDVLGNKTWDRETRDLTGNGKDLRMTLAHIKDQKEVLQ
SSDLGHERQEFLEGRCHEDQDTLSRYACRLPAGTTAISRIQRSP
EENSAPHGECHPQRGLRGQHHRDMQGFQLLSPEYHTDLASGWGI
FEPRHPAVGGCPA
MICA-B1
AA1-AA265
SEQ ID NO 2
EPHSLRYNLTVLSGDGSVQSGFLAEVHLDGQPFLRCDRQKCRAK
PQGQWAEDVLGNKTWDRETRDLTGNGKDLRMTLAHIKDQKEVLQ
SSDLGHERQEFLEGRCHEDQDTLSRYACRLPAGTTAISKIRRSP
EENSAPHGECHPQRGLEGQHYRDMQGFWLLSLEYHTELASGWGK
VLVLQSHWQTFHVSAVAAAAAAAAAIFVIIIFYVCCCKKKTSAA
EGPELVSLQVLDQHPVGTSDHRDATQLGFQPLMSDLGSTGSTEG
A
(MICA-B2)
AA1-AA272
SEQ ID NO 3
EPHSLRYNLTVLSGDGSVQSGFLAEVHLDGQPFLRCDRQKCRAK
PQGQWAEDVLGNKTWDRETRDLTGNGKDLRMTLAHIKDQKEGLH
SLQEIRVCEIHEDNSTRSSQHFYYDGELFLSQNLETEEWTMPQS
SRAQTLAMNVRNFLKEDAMKTKTHYHAMHADCLQELRRYLKSGV
VLRRTGKVLVLQSHWQTFHVSAVAAAAAAAAAIFVIIIFYVCCC
KKKTSAAEGPELVSLQVLDQHPVGTSDHRDATQLGFQPLMSDLG
STGSTEGA
(MICA-C)
AA1-AA175
SEQ ID NO 4
EPHSLRYNLTVLSGDGSVQSGFLAEVHLDGQPFLRCDRQKCRAK
PQGQWAEDVLGNKTWDRETRDLTGNGKDLRMTLAHIKDQKEVLQ
SSDLGHERQEFLEGRCHEDQDTLSRYACRLPAGTTAISKIRRSP
EENRSAGASESLADIPCFCCCCCCCCCCCYFCYYYFLRLLL
(MICA-D)
AA1-AA176
SEQ ID NO 5
EPHSLRYNLTVLSGDGSVQSGFLAEVHLDGQPFLRCDRQKCRAK
PQGQWAEDVLGNKTWDRETRDLTGNGKDLRMTLAHIKDQKEGKV
LVLQSHWQTFHVSAVAAAAAAAAAIFVIIIFYVCCCKKKTSAAE
GPELVSLQVLDQHPVGTSDHRDATQLGFQPLMSDLGSTGSTEGA

By “purified” and “isolated” it is meant, when referring to a polypeptide or a nucleotide sequence, that the indicated molecule is present in the substantial absence of other biological macromolecules of the same type. The term “purified” as used herein typically means at least 75% by weight, more typically at least 85% by weight, still typically at least 95% by weight, and most typically at least 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

A further aspect of the invention, to a function conservative variant of a polypeptide as above described wherein the variant has at least 80% of identity with a sequence selected from the group consisting of SEQ ID NO:1 (MICA-A), SEQ ID NO:2 (MICA-B1), SEQ ID NO:3 (MICA-B2); SEQ ID NO:4 (MICA-C) and SEQ ID NO:5 (MICA-D).

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 80% to 99% as determined according to an alignment scheme such as by the Cluster Method, wherein similarity is based on the MEGALIGN algorithm. Typically, the function conservative variant has 80, 81, 82, 83, 84, 84, 85, 86, 87, 88, 89, 90, 91, 92, 93, 94, 95, 96, 97, 98, 99% of identity with a sequence selected from the group consisting of SEQ ID NO:1 (MICA-A), SEQ ID NO:2 (MICA-B1), SEQ ID NO:3 (MICA-B2); SEQ ID NO:4 (MICA-C) and SEQ ID NO:5 (MICA-D). Two amino acid sequences are “substantially homologous” or “substantially similar” when greater than 80%, typically greater than 85%, typically greater than 90% of the amino acids are identical, or greater than about 90%, typically grater than 95%, are similar (functionally identical). Typically, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

In one embodiment a variant of SEQ ID NO:1 (MICA-A) comprises an amino acid sequence having at least 80% of identity with the sequence ranging from the amino acid residue at position 86 to the amino acid residue at position 189 in SEQ ID NO:1.

In one embodiment a variant of SEQ ID NO:2 (MICA-B1) comprises an amino acid sequence having at least 80% of identity with the sequence ranging from the amino acid residue at position 86 to the amino acid residue at position 174 in SEQ ID NO:2.

In one embodiment a variant of SEQ ID NO:4 (MICA-C) comprises an amino acid sequence having at least 80% of identity with the sequence ranging from the amino acid residue at position 86 to the amino acid residue at position 175 in SEQ ID NO:4.

A further aspect of the invention relates to a fragment of a polypeptide as above described.

In one embodiment, the fragment comprises all or a portion of the extracellular domains of the polypeptides characterized by SEQ ID NO:2 (MICA-B1), SEQ ID NO:3 (MICA-B2) or SEQ ID NO:5 (MICA-D). Accordingly, the variants comprise soluble form of the polypeptide characterized by SEQ ID NO:2 (MICA-B1), SEQ ID NO:3 (MICA-B2) or SEQ ID NO:5 (MICA-D). A suitable soluble form of these polypeptide might comprise, for example, a truncated form of the polypeptide from which the transmembrane domain and the cytoplasmic domain have been removed by chemical, proteolytic or recombinant methods. In one embodiment, the fragment is at least 80, 81, 82, 83, 84, 84, 85, 86, 87, 88, 89, 90, 91, 92, 93, 94, 95, 96, 97, 98, 99% homologous to the extracellular domains of the polypeptides characterized by SEQ ID NO:2 (MICA-B1), SEQ ID NO:3 (MICA-B2) or SEQ ID NO:5 (MICA-D).

In one embodiment a fragment of SEQ ID NO:1 (MICA-A) comprises an amino acid sequence ranging from the amino acid residue at position 86 to the amino acid residue at position 189 in SEQ ID NO:1.

In one embodiment a fragment of SEQ ID NO:2 (MICA-B1) comprises an amino acid sequence ranging from the amino acid residue at position 86 to the amino acid residue at position 174 in SEQ ID NO:2.

In one embodiment a fragment of SEQ ID NO:4 (MICA-C) comprises an amino acid sequence ranging from the amino acid residue at position 86 to the amino acid residue at position 175 in SEQ ID NO:4.

A further aspect of the invention relates to a fusion protein comprising a polypeptide as above described fused to a heterologous polypeptide (i.e. a polypeptide that do not derive from a polypeptide of the invention).

As used herein, a fusion protein” comprises all or part (typically biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the N-terminus or C-terminus of the polypeptide of the invention.

One useful fusion protein is a GST fusion protein in which the polypeptide of the invention is fused to the C-terminus of GST sequences. Such fusion proteins can facilitate the purification of a recombinant polypeptide of the invention.

In one embodiment, the fusion protein contains a heterologous signal sequence at its N-terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products). In one embodiment, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain. Even more the signal sequence can represent a sequence that will facilitate the production of the protein of interest in particular cell interest. For example, the polypeptide of the invention may be fused to a sequence that will drive the expression of the polypeptide in the exosomes or microparticles (or other vesicles)

In one embodiment, the fusion protein according to the invention is an immunoadhesin.

As used herein, the term “immunoadhesin” designates antibody-like molecules which combine the binding specificity of a heterologous protein (an “adhesin” which is able to bind to NKG2D) with the effector functions of immunoglobulin constant domains. Structurally, the immunoadhesins comprise a fusion of an amino acid sequence with the desired binding specificity to NKG2D (i.e., is “heterologous”), and an immunoglobulin constant domain sequence. The adhesin part of an immunoadhesin molecule typically is a contiguous amino acid sequence comprising at least the binding site of a receptor or a ligand. In one embodiment, the adhesin comprises the polypeptides characterized by SEQ ID NO:1 (MICA-A) or SEQ ID NO:4 (MICA-C). In one embodiment, the adhesin comprises the soluble form of the polypeptide characterized by SEQ ID NO:2 (MICA-B1), SEQ ID NO:3 (MICA-B2) or SEQ ID NO:5 (MICA-D). The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The immunoglobulin sequence typically, but not necessarily, is an immunoglobulin constant domain (Fc region). Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use.

In one embodiment, the Fc region is a native sequence Fc region. In one embodiment, the Fc region is a variant Fc region. In still another embodiment, the Fc region is a functional Fc region. As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The adhesion portion and the immunoglobulin sequence portion of the immunoadhesin may be linked by a minimal linker. The immunoglobulin sequence typically, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but typically IgG1 or IgG3.

The polypeptides of the invention, fragments thereof and fusion proteins according to the invention can exhibit post-translational modifications, including, but not limited to glycosylations, (e.g., N-linked or O-linked glycosylations), myristylations, palmitylations, acetylations and phosphorylations (e.g., serine/threonine or tyrosine).

In some embodiments, the polypeptide of the invention and the immunoglobulin sequence portion of the immunoadhesin are linked by a minimal linker. As used herein, the term “linker” refers to a sequence of at least one amino acid that links the polypeptide of the invention and the immunoglobulin sequence portion. Such a linker may be useful to prevent steric hindrances. In some embodiments, the linker has 4; 5; 6; 7; 8; 9; 10; 11; 12; 13; 14; 15; 16; 17; 18; 19; 20; 21; 22; 23; 24; 25; 26; 27; 28; 29; 30 amino acid residues. However, the upper limit is not critical but is chosen for reasons of convenience regarding e.g. biopharmaceutical production of such polypeptides. The linker sequence may be a naturally occurring sequence or a non-naturally occurring sequence. If used for therapeutical purposes, the linker is preferably non-immunogenic in the subject to which the immunoadhesin is administered. One useful group of linker sequences are linkers derived from the hinge region of heavy chain antibodies as described in WO 96/34103 and WO 94/04678. Other examples are poly-alanine linker sequences.

The polypeptides of the invention, fragments thereof and fusion proteins according to the invention may be produced by any technique known per se in the art, such as, without limitation, any chemical, biological, genetic or enzymatic technique, either alone or in combination. For example, knowing the amino acid sequence of the desired sequence, one skilled in the art can readily produce said polypeptides, by standard techniques for production of polypeptides. For instance, they can be synthesized using well-known solid phase method, typically using a commercially available peptide synthesis apparatus (such as that made by Applied Biosystems, Foster City, Calif.) and following the manufacturer's instructions. Alternatively, the polypeptides of the invention can be synthesized by recombinant DNA techniques as is now well-known in the art. For example, these fragments can be obtained as DNA expression products after incorporation of DNA sequences encoding the desired polypeptide into expression vectors and introduction of such vectors into suitable eukaryotic or prokaryotic hosts that will express the desired polypeptide, from which they can be later isolated using well-known techniques.

One aspect of the invention pertains to isolated nucleic acid molecules that encode a polypeptide of the invention, a fragment thereof or a fusion protein of the invention, as well as nucleic acid molecules sufficient for use as hybridization probes to identify nucleic acid molecules encoding said polypeptides and fragments of such nucleic acid molecules suitable for use as PCR primers for the amplification or mutation of nucleic acid molecules.

In particular embodiment, the invention relates to an isolated nucleic acid molecule nucleic acid molecule comprising a nucleotide sequence which is at least 80% identical to the nucleotide sequence of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15.

(MICA-A)
Transcript (945 bp)
SEQ ID NO 6
GAGCCCCACAGTCTTCGTTATAACCTCACGGTGCTGTCCGGGGA
TGGATCTGTGCAGTCAGGGTTTCTCGCTGAGGTACATCTGGATG
GTCAGCCCTTCCTGCGCTGTGACAGGCAGAAATGCAGGGCAAAG
CCCCAGGGACAGTGGGCAGAAGATGTCCTGGGAAATAAGACATG
GGACAGAGAGACCAGGGACTTGACAGGGAACGGAAAGGACCTCA
GGATGACCCTGGCTCATATCAAGGACCAGAAAGAAGTCCTCCAG
AGCTCAGACCTTGGCCATGAACGTCAGGAATTTCTTGAAGGAAG
ATGCCATGAAGACCAAGACACACTATCACGCTATGCATGCAGAC
TGCCTGCAGGAACTACGGCGATATCTAGAATCCAGCGTAGTCCT
GAGGAGAACAGTGCCCCCCATGGTGAATGTCACCCGCAGCGAGG
CCTCAGAGGGCAACATCACCGTGACATGCAGGGCTTCCAGCTTC
TATCCCCGGAATATCATACTGACCTGGCGTCAGGATGGGGTATC
TTTGAGCCACGACACCCAGCAGTGGGGGGATGTCCTGCCTGATG
GGAATGGAACCTACCAGACCTGGGTGGCCACCAGGATTTGCCGA
GGAGAGGAGCAGAGGTTCACCTGCTACATGGAACACAGCGGGAA
TCACAGCACTCACCCTGTGCCTCTGGGAAAGTGCTGTGCTTCAG
AGTCATTGGCAGACATTCCATGTTCTGCTGTGCTGCTGCTGCTG
CTATTTTTGTTATTATTATTTTCTATGTCCGTTGTTGTAAGAAG
AAAACATCAGCTGCAGAGGGTCCAGAGCTCGTGAGCCTGCAGGT
CCTGGATCAACACCCAGTTGGGACGAGTGACCACAGGGATGCCA
CACAGCTCGGATTTCAGCCTCTGATGTCAGCTCTTGGGTCCACT
GGCTCCACTGAGGGCGCCTAG
(MICA-B1)
Transcript (797 bp)
SEQ ID NO 7
GAGCCCCACAGTCTTCGTTATAACCTCACGGTGCTGTCCGGGGA
TGGATCTGTGCAGTCAGGGTTTCTCGCTGAGGTACATCTGGATG
GTCAGCCCTTCCTGCGCTGTGACAGGCAGAAATGCAGGGCAAAG
CCCCAGGGACAGTGGGCAGAAGATGTCCTGGGAAATAAGACATG
GGACAGAGAGACCAGGGACTTGACAGGGAACGGAAAGGACCTCA
GGATGACCCTGGCTCATATCAAGGACCAGAAAGAAGTCCTCCAG
AGCTCAGACCTTGGCCATGAACGTCAGGAATTTCTTGAAGGAAG
ATGCCATGAAGACCAAGACACACTATCACGCTATGCATGCAGAC
TGCCTGCAGGAACTACGGCGATATCTAAAATCCGGCGTAGTCCT
GAGGAGAACAGTGCCCCCCATGGTGAATGTCACCCGCAGCGAGG
CCTCAGAGGGCAACATTACCGTGACATGCAGGGCTTCTGGCTTC
TATCCCTGGAATATCACACTGAGCTGGCGTCAGGATGGGGGAAA
GTGCTGGTGCTTCAGAGTCATTGGCAGACATTCCATGTTTCTGC
TGTTGCTGCTGCTGCTGCTGCTGCTGCTGCTATTTTTGTTATTA
TTATTTTCTACGTCTGTTGTTGTAAGAAGAAAACATCAGCTGCA
GAGGGTCCAGAGCTCGTGAGCCTGCAGTCCTGGATCAACACCCA
GTTGGGACGAGTGACCACAGGGATGCCACACAGCTCGGATTTCA
GCCTCTGATGTCAGATCTTGGGTCCACTGGCTCCACTGAGGGCG
CCTAG
(MICA-B2)
Transcript (819 bp)
SEQ ID NO 8
GAGCCCCACAGTCTTCGTTATAACCTCACGGTGCTGTCCGGGGA
TGGATCTGTGCAGTCAGGGTTTCTTGCTGAGGTACATCTGGATG
GTCAGCCCTTCCTGCGCTATGACAGGCAGAAATGCAGGGCAAAG
CCCCAGGGACAGTGGGCAGAAGATGTCCTGGGAAATAAGACATG
GGACAGAGAGACCAGSGACTTGACAGGGAACGGAAAGGACCTCA
GGATGACCCTGGCTCATATCAAGGACCAGAAAGAAGGCTTGCAT
TCCCTCCAGGAGATTAGGGTCTGTGAGATCCATGAAGACAACAG
CACCAGGAGCTCCCAGCATTTCTACTACGATGGGGAGCTCTTCC
TCTCCCAAAACCTGGAGACTGAGGAATGGACAGTGCCCCAGTCC
TCCAGAGCTCAGACCTTGGCCATGAACGTCAGGAATTTCTTGAA
GGAAGATGCCATGAAGACCAAGACACACTATCACGCTATGCATG
CAGACTGCCTGCAGGAACTACGGCGATATCTAGAATCCAGCGTA
GTCCTGAGGAGAACAGGGAAAGTGCTGGTGCTTCAGAGTCATTG
GCAGACATTCCATGTTTCTGCTGTTGCTGCTGCTGCTGCTGCTG
CTGCTGCTATTTTTGTTATTATTATTTTCTACGTCTGTTGTTGT
AAGAAGAAAACATCAGCTGCAGAGGGTCCAGAGCTCGTGAGCCT
GCAGGTCCTGGATCAACACCCAGTTGGGACGAGTGACCACAGGG
ATGCCACACAGCTCGGATTTCAGCCTCTGATGTCAGATCTTGGG
TCCACTGGCTCCACTGAGGGCGCCTAG
(MICA-C)
Transcript (682 bp)
SEQ ID NO 9
GAGCCCCACAGTCTTCGTTATAACCTCACGGTGCTGTCCGGGGA
TGGATCTGTGCAGTCAGGGTTTCTCGCTGAGGTACATCTGGATG
GTCAGCCCTTCCTGCGCTGTGACAGGCAGAAATGCAGGGCAAAG
CCCCAGGGACAGTGGGCAGAAGATGTCCTGGGAAATAAGACATG
GGACAGAGAGACCAGGGACTTGACAGGGAACGGAAAGGACCTCA
GGATGACCCTGGCTCATATCAAGGACCAGAAAGAAGTCCTCCAG
AGCTCAGACCTTGGCCATGAACGTCAGGAATTTCTTGAAGGAAG
ATGCCATGAAGACCAAGACACACTATCACGCTATGCATGCAGAC
TGCCTGCAGGAACTACGGCGATATCTAAAATCCGGCGTAGTCCT
GAGGAGAACAGGGAAAGTGCTGGTGCTTCAGAGTCATTGGCAGA
CATTCCATGTTTCTGCTGTTGCTGCTGCTGCTGCTGCTGCTGCT
GCTATTTTTGTTATTATTATTTTCTACGTCTGTTGTTGTAAGAA
GAAAACATCAGCTGCAGAGGGTCCAGAGCTCGTGAGCCTGCAGG
TCCTGGATCAACACCCAGTTGGGACGAGTGACCACAGGGATGCC
ACACAGCTCGGATTTCAGCCTCTGATGTCAGATCTTGGGTCCAC
TGGCTCCACTGAGGGCGCCTAG
(MICA-D)
Transcript (531 bp)
SEQ ID NO 10
GAGCCCCACAGTCTTCGTTATAACCTCACGGTGCTGTCCTGGGA
TGGATCTGTGCAGTCAGGGTTTCTTGCTGAGGTACATCTGGATG
GTCAGCCCTTCCTGCGCTATGACAGGCAGAAATGCAGGGCAAAG
CCCCAGGGACAGTGGGCAGAAGATGTCCTGGGAAATAAGACATG
GGACAGAGAGACCAGGGACTTGACAGGGAACGGAAAGGACCTCA
GGATGACCCTGGCTCATATCAAGGACCAGAAAGAAGGGAAAGTG
CTGGTGCTTCAGAGTCATTGGCAGACATTCCATGTTTCTGCTGT
TGCTGCTGCTGCTGCTGCTGCTGCTGCTATTTTTGTTATTATTA
TTTTCTATGTCCGTTGTTGTAAGAAGAAAACATCAGCTGCAGAG
GGTCCAGAGCTCGTGAGCCTGCAGGTCCTGGATCAACACCCAGT
TGGGACGAGTGACCACAGGGATGCCACACAGCTCGGATTTCAGC
CTCTGATGTCAGATCTTGGGTCCACTGGCTCCACTGAGGGCGCC
TAG
(MICA-A)
cDNA: exon1-exon6 (1188 bp)
SEQ ID NO 11.
ATGGGGCTGGGCCCGGTCTTCCTGCTTCTGGCTGGCATCTTCCC
TTTTGCACCTCCGGGAGCTGCTGCTGAGCCCCACAGTCTTCGTT
ATAACCTCACGGTGCTGTCCGGGGATGGATCTGTGCAGTCAGGG
TTTCTCGCTGAGGTACATCTGGATGGTCAGCCCTTCCTGCGCTG
TGACAGGCAGAAATGCAGGGCAAAGCCCCAGGGACAGTGGGCAG
AAGATGTCCTGGGAAATAAGACATGGGACAGAGAGACCAGGGAC
TTGACAGGGAACGGAAAGGACCTCAGGATGACCCTGGCTCATAT
CAAGGACCAGAAAGAAGTCCTCCAGAGCTCAGACCTTGGCCATG
AACGTCAGGAATTTCTTGAAGGAAGATGCCATGAAGACCAAGAC
ACACTATCACGCTATGCATGCAGACTGCCTGCAGGAACTACGGC
GATATCTAGAATCCAGCGTAGTCCTGAGGAGAACAGTGCCCCCC
ATGGTGAATGTCACCCGCAGCGAGGCCTCAGAGGGCAACATCAC
CGTGACATGCAGGGCTTCCAGCTTCTATCCCCGGAATATCATAC
TGACCTGGCGTCAGGATGGGGTATCTTTGAGCCACGACACCCAG
CAGTGGGGGGATGTCCTGCCTGATGGGAATGGAACCTACCAGAC
CTGGGTGGCCACCAGGATTTGCCGAGGAGAGGAGCAGAGGTTCA
CCTGCTACATGGAACACAGCGGGAATCACAGCACTCACCCTGTG
CCTCTGGGAAAGTGCTGTGCTTCAGAGTCATTGGCAGACATTCC
ATGTTCTGCTGTGCTGCTGCTGCTGCTATTTTTGTTATTATTAT
TTTCTATGTCCGTTGTTGTAAGAAGAAAACATCAGCTGCAGAGG
GTCCAGAGCTCGTGAGCCTGCAGGTCCTGGATCAACACCCAGTT
GGGACGAGTGACCACAGGGATGCCACACAGCTCGGATTTCAGCC
TCTGATGTCAGCTCTTGGGTCCACTGGCTCCACTGAGGGCGCCT
AGACTCTACAGCCAGGCGGCTGGAATTGAATTCCCTGCCTGGAT
CTCACAAGCACTTTCCCTCTTGGTGCCTCAGTTTCCTGACCTAT
GAAACAGAGAAAATAAAAGCACTTATTTATTGTTGTTGGAGGCT
GCAAAATGTTAGTAGATATGAGGCATTTGCAGCTGTGCCATATT
(MICA-B1)
cDNA: exon1-exon6 (1040 bp)
SEQ ID NO 12
ATGGGGCTGGGCCCGGTCTTCCTGCTTCTGGCTGGCATCTTCCC
TTTTGCACCTCCGGGAGCTGCTGCTGAGCCCCACAGTCTTCGTT
ATAACCTCACGGTGCTGTCCGGGGATGGATCTGTGCAGTCAGGG
TTTCTCGCTGAGGTACATCTGGATGGTCAGCCCTTCCTGCGCTG
TGACAGGCAGAAATGCAGGGCAAAGCCCCAGGGACAGTGGGCAG
AAGATGTCCTGGGAAATAAGACATGGGACAGAGAGACCAGGGAC
TTGACAGGGAACGGAAAGGACCTCAGGATGACCCTGGCTCATAT
CAAGGACCAGAAAGAAGTCCTCCAGAGCTCAGACCTTGGCCATG
AACGTCAGGAATTTCTTGAAGGAAGATGCCATGAAGACCAAGAC
ACACTATCACGCTATGCATGCAGACTGCCTGCAGGAACTACGGC
GATATCTAAAATCCGGCGTAGTCCTGAGGAGAACAGTGCCCCCC
ATGGTGAATGTCACCCGCAGCGAGGCCTCAGAGGGCAACATTAC
CGTGACATGCAGGGCTTCTGGCTTCTATCCCTGGAATATCACAC
TGAGCTGGCGTCAGGATGGGGGAAAGTGCTGGTGCTTCAGAGTC
ATTGGCAGACATTCCATGTTTCTGCTGTTGCTGCTGCTGCTGCT
GCTGCTGCTGCTATTTTTGTTATTATTATTTTCTACGTCTGTTG
TTGTAAGAAGAAAACATCAGCTGCAGAGGGTCCAGAGCTCGTGA
GCCTGCAGTCCTGGATCAACACCCAGTTGGGACGAGTGACCACA
GGGATGCCACACAGCTCGGATTTCAGCCTCTGATGTCAGATCTT
GGGTCCACTGGCTCCACTGAGGGCGCCTAGACTCTACAGCCAGG
CGGCTGGAATTGAATTCCCTGCCTGGATCTCACAAGCACTTTCC
CTCTTGGTGCCTCAGTTTCCTGACCTATGAAACAGAGAAAATAA
AAGCACTTATTTATTGTTGTTGGAGGCTGCAAAATGTTAGTAGA
TATGAGGCATTTGCAGCTGTGCCATATT
(MICA-B2)
cDNA: exon1-exon6 (1062 bp)
SEQ ID NO 13
ATGGGGCTGGGCCCGGTCTTCCTGCTTCTGGCTGGCATCTTCCC
TTTTGCACCTCCGGGAGCTGCTGCTGAGCCCCACAGTCTTCGTT
ATAACCTCACGGTGCTGTCCGGGGATGGATCTGTGCAGTCAGGG
TTTCTTGCTGAGGTACATCTGGATGGTCAGCCCTTCCTGCGCTA
TGACAGGCAGAAATGCAGGGCAAAGCCCCAGGGACAGTGGGCAG
AAGATGTCCTGGGAAATAAGACATGGGACAGAGAGACCAGSGAC
TTGACAGGGAACGGAAAGGACCTCAGGATGACCCTGGCTCATAT
CAAGGACCAGAAAGAAGGCTTGCATTCCCTCCAGGAGATTAGGG
TCTGTGAGATCCATGAAGACAACAGCACCAGGAGCTCCCAGCAT
TTCTACTACGATGGGGAGCTCTTCCTCTCCCAAAACCTGGAGAC
TGAGGAATGGACAGTGCCCCAGTCCTCCAGAGCTCAGACCTTGG
CCATGAACGTCAGGAATTTCTTGAAGGAAGATGCCATGAAGACC
AAGACACACTATCACGCTATGCATGCAGACTGCCTGCAGGAACT
ACGGCGATATCTAGAATCCAGCGTAGTCCTGAGGAGAACAGGGA
AAGTGCTGGTGCTTCAGAGTCATTGGCAGACATTCCATGTTTCT
GCTGTTGCTGCTGCTGCTGCTGCTGCTGCTGCTATTTTTGTTAT
TATTATTTTCTACGTCTGTTGTTGTAAGAAGAAAACATCAGCTG
CAGAGGGTCCAGAGCTCGTGAGCCTGCAGGTCCTGGATCAACAC
CCAGTTGGGACGAGTGACCACAGGGATGCCACACAGCTCGGATT
TCAGCCTCTGATGTCAGATCTTGGGTCCACTGGCTCCACTGAGG
GCGCCTAGACTCTACAGCCAGGCGGCTGGAATTGAATTCCCTGC
CTGGATCTCACAAGCACTTTCCCTCTTGGTGCCTCAGTTTCCTG
ACCTATGAAACAGAGAAAATAAAAGCACTTATTTATTGTTGTTG
GAGGCTGCAAAATGTTAGTAGATATGAGGCATTTGCAGCTGTGC
CATATT
(MICA-C)
cDNA: exon1-exon6 (925 bp)
SEQ ID NO 14
ATGGGGCTGGGCCCGGTCTTCCTGCTTCTGGCTGGCATCTTCCC
TTTTGCACCTCCGGGAGCTGCTGCTGAGCCCCACAGTCTTCGTT
ATAACCTCACGGTGCTGTCCGGGGATGGATCTGTGCAGTCAGGG
TTTCTCGCTGAGGTACATCTGGATGGTCAGCCCTTCCTGCGCTG
TGACAGGCAGAAATGCAGGGCAAAGCCCCAGGGACAGTGGGCAG
AAGATGTCCTGGGAAATAAGACATGGGACAGAGAGACCAGGGAC
TTGACAGGGAACGGAAAGGACCTCAGGATGACCCTGGCTCATAT
CAAGGACCAGAAAGAAGTCCTCCAGAGCTCAGACCTTGGCCATG
AACGTCAGGAATTTCTTGAAGGAAGATGCCATGAAGACCAAGAC
ACACTATCACGCTATGCATGCAGACTGCCTGCAGGAACTACGGC
GATATCTAAAATCCGGCGTAGTCCTGAGGAGAACAGGGAAAGTG
CTGGTGCTTCAGAGTCATTGGCAGACATTCCATGTTTCTGCTGT
TGCTGCTGCTGCTGCTGCTGCTGCTGCTATTTTTGTTATTATTA
TTTTCTACGTCTGTTGTTGTAAGAAGAAAACATCAGCTGCAGAG
GGTCCAGAGCTCGTGAGCCTGCAGGTCCTGGATCAACACCCAGT
TGGGACGAGTGACCACAGGGATGCCACACAGCTCGGATTTCAGC
CTCTGATGTCAGATCTTGGGTCCACTGGCTCCACTGAGGGCGCC
TAGACTCTACAGCCAGGCGGCTGGAATTGAATTCCCTGCCTGGA
TCTCACAAGCACTTTCCCTCTTGGTGCCTCAGTTTCCTGACCTA
TGAAACAGAGAAAATAAAAGCACTTATTTATTGTTGTTGGAGGC
TGCAAAATGTTAGTAGATATGAGGCATTTGCAGCTGTGCCATAT
T
(MICA-D)
cDNA: exon1-exon6 (774 bp)
SEQ ID NO 15
ATGGGGCTGGGCCCGGTCTTCCTGCTTCTGGCTGGCATCTTCCC
TTTTGCACCTCCGGGAGCTGCTGCTGAGCCCCACAGTCTTCGTT
ATAACCTCACGGTGCTGTCCTGGGATGGATCTGTGCAGTCAGGG
TTTCTTGCTGAGGTACATCTGGATGGTCAGCCCTTCCTGCGCTA
TGACAGGCAGAAATGCAGGGCAAAGCCCCAGGGACAGTGGGCAG
AAGATGTCCTGGGAAATAAGACATGGGACAGAGAGACCAGGGAC
TTGACAGGGAACGGAAAGGACCTCAGGATGACCCTGGCTCATAT
CAAGGACCAGAAAGAAGGGAAAGTGCTGGTGCTTCAGAGTCATT
GGCAGACATTCCATGTTTCTGCTGTTGCTGCTGCTGCTGCTGCT
GCTGCTGCTATTTTTGTTATTATTATTTTCTATGTCCGTTGTTG
TAAGAAGAAAACATCAGCTGCAGAGGGTCCAGAGCTCGTGAGCC
TGCAGGTCCTGGATCAACACCCAgTTGGGACGAGTGACCACAGG
GATGCCACACAGCTCGGATTTCAGCCTCTGATgTcAGATcTTGG
gTCCactGGcTCCACTGAGGGCGCCTAGACTCTACAGCCAGGCG
GCTGGAATTGAATTCCCTGCCTGGATCTCACAAGCACTTTCCCT
CTTGGTGCCTCAGTTTCCTGACCTATGAAACAGAGAAAATAAAA
GCACTTATTTATTGTTGTTGGAGGCTGCAAAATGTTAGTAGATA
TGAGGCATTTGCAGCTGTGCCATATT

In one embodiment the isolated nucleic acid molecule of the invention comprises a nucleotide sequence which at least 85%, 95%, or 98% identical to the nucleotide sequence of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15.

In an further embodiment, a nucleic acid molecule of the invention consists of the nucleotide sequence of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15.

A nucleic acid molecule of the present invention can be isolated using standard molecular biology techniques and the sequence information provided herein. Using all or a portion of the nucleic acid sequences of the invention as a hybridization probe, nucleic acid molecules of the invention can be isolated using standard hybridization and cloning techniques (e.g., as described in Sambrook et al., eds., MolecularCloning: A Laboratory Manual, 2nd ed., Cold Spring Harbor Laboratory, Cold Spring Harbor Laboratory Press, Cold Spring Harbor, N. Y., 1989).

A nucleic acid molecule of the invention can be amplified using cDNA, mRNA or genomic DNA as a template and appropriate oligonucleotide primers according to standard methods. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In one embodiment, an isolated nucleic acid molecule of the invention comprises a nucleic acid molecule which is complementary of the nucleotide sequence of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence encoding a full length polypeptide of the invention for example, a fragment which can be used as a probe or primer or a fragment encoding a biologically active portion of a polypeptide of the invention. The nucleotide sequence determined from the cloning one gene allows for the generation of probes and primers designed for use in identifying and/or cloning homologues in other cell types, e.g., from other tissues, as well as homologues from other mammals. The probe/primer typically comprises substantially purified oligonucleotide.

In one embodiment, the oligonucleotide comprises a region of nucleotide sequence that hybridizes under stringent conditions to at least about 12, typically about 25, more typically about 50, 75, 100, 125, 150, 175, 200, 250, 300, 350 or 400 consecutive nucleotides of the sense or anti-sense sequence of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15.

In one embodiment, the oligonucleotide comprises or consists of a sequence selected from the group consisting of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, and SEQ ID NO:25.

MICA-A Primers
(MICA-A)
SEQ ID NO 16
GGACAGTGGGCAGAAGATGTC
(MICA-A)
SEQ ID NO 17
GCTCTGGAGGACTTCTTTCTGG
MICA-B1 primers
(MICA-B1)
SEQ ID NO 18
GCCATGAAGACCAAGACACACTA
(MICA-B1)
SEQ ID NO 19
ACCAGCACTTTCCCCCATC
MICA-B2 primers
(MICA-B2)
SEQ ID NO 20
GCCATGAAGACCAAGACACACTA
(MICA-B2)
SEQ ID NO 21
CCAGCACTTTCCCTGTTCTCC
MICA-C primers
(MICA-C)
SEQ ID NO 22
CCAGAAAGAAGTCCTCCAGAGC
MICA-C)
SEQ ID NO 23
GCACTTTCCCTGTTCTCCTCA
MICA-D primers
(MICA-D)
SEQ ID NO 24
TGGATGGTCAGCCCTTCCT
(MICA-D)
SEQ ID NO 25
GCACTTTCCCTTCTTTCTGGTC

Probes based on the sequence of a nucleic acid molecule of the invention can be used to detect transcripts or genomic sequences encoding the same protein molecule encoded by a selected nucleic acid molecule. The probe comprises a label group attached thereto, e.g., a radioisotope, a fluorescent compound, an enzyme, or an enzyme co-factor. Such probes can be used as part of a diagnostic test kit for identifying cells or tissues which express or not the protein, such as by measuring levels of a nucleic acid molecule encoding the protein in a sample of cells from a subject, e.g., detecting mRNA levels or determining whether a gene encoding the protein has been mutated or deleted.

The invention also pertains to the couple of oligonucleotide primers SEQ ID NO:16 and SEQ ID NO:17 for amplifying the nucleic acid molecule consisting of SEQ ID NO:6 (MICA-A).

The invention also pertains to the couple of oligonucleotide primers SEQ ID NO:18 and SEQ ID NO:19 for amplifying the nucleic acid molecule consisting of SEQ ID NO:7 (MICA-B1).

The invention also pertains to the couple of oligonucleotide primers SEQ ID NO:20 and SEQ ID NO:21 for amplifying the nucleic acid molecule consisting of SEQ ID NO:8 (MICA-B2).

The invention also pertains to the couple of oligonucleotide primers SEQ ID NO:22 and SEQ ID NO:23 for amplifying the nucleic acid molecule consisting of SEQ ID NO:9 (MICA-C).

The invention also pertains to the couple of oligonucleotide primers SEQ ID NO:24 and SEQ ID NO:25 for amplifying the nucleic acid molecule consisting of SEQ ID NO:10 (MICA-D).

A nucleic acid fragment encoding a biologically active portion of a polypeptide of the invention can be prepared by isolating a portion of any of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) expressing the encoded portion of the polypeptide protein (e. g., by recombinant expression in vitro).

The invention further encompasses nucleic acid molecules that differ from the nucleotide sequence of due to degeneracy of the genetic code and thus encode the same protein as that encoded by the nucleotide sequence of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15.

Accordingly, in one embodiment, an isolated nucleic acid molecule of the invention is at least 100, 200, 300, 400, or 500 contiguous nucleotides in length and hybridizes under stringent conditions to the nucleic acid molecule comprising the nucleotide sequence, typically the coding sequence, of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15 or a complement thereof.

The invention further encompasses nucleic acid molecules that differ from the nucleotide sequence of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15 due to degeneracy of the genetic code and thus encode the same protein as that encoded by the nucleotide sequence of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15.

In addition to the nucleotide sequences of the invention, it will be appreciated by those skilled in the art that DNA sequence polymorphisms that can lead to changes in the amino acid sequence may exist within a population (e. g, the human population). Such genetic polymorphisms may exist among individuals within a population due to natural allelic variation. An allele is one of a group of genes which occur alternatively at a given genetic locus. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In one embodiment, the Single nucleotide polymorphism (SNPs) that characterize the MICA *015 and MICA*017 and that are associated with the novel MICA alternative transcripts and isoforms include but are not limited to Rs41558312 (A/G), Rs41556715 (A-G), Rs1051792 (A/G), Rs199503730 (-G), Rs61738275 (C/T), Rs41558418 (-G), Rs41553217 (A/G).

Moreover, nucleic acid molecules encoding proteins of the invention from other species (homologues), which have a nucleotide sequence which differs from that of rat protein described herein are intended to be within the scope of the invention.

Nucleic acid molecules corresponding to natural allelic variants and homologues of a cDNA of the invention can be isolated based on their identity to the human nucleic acid molecule disclosed herein using the human cDNAs, or a portion thereof, as a hybridization probe according to standard hybridization techniques under stringent hybridization conditions.

In addition to naturally-occurring allelic variants of a nucleic acid molecule of the invention sequence that may exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologues of various species may be non-essential for activity and thus would be likely targets for alteration. Alternatively, amino acid residues that are conserved among the homologues of various species (e.g., mouse and human) may be essential for activity and thus would not be likely targets for alteration.

Mutations can be introduced by standard techniques, such as site-directed mutagenesis and PCR-mediated mutagenesis. Preferably, conservative amino acid substitutions are made at one or more predicted non-essential amino acid residues.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity.

Following mutagenesis, the encoded protein can be expressed recombinantly and the activity of the protein can be determined.

In a preferred embodiment, a mutant polypeptide that is a variant of a polypeptide of the invention can be assayed for: (1) the ability to form protein: protein interactions with proteins in a signaling pathway of the polypeptide of the invention; (2) the ability to bind a ligand of the polypeptide of the invention; (3) the ability to bind to an intracellular target protein of the polypeptide of the invention; or (4) the ability to activate an intracellular signalling molecule activated by the polypeptide of the invention.

The present invention encompasses antisense nucleic acid molecules, i. e., molecules which are complementary to a sense nucleic acid encoding a polypeptide of the invention, e.g., complementary to the coding strand of a double-stranded cDNA molecule or complementary to an mRNA sequence. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′untranslated regions”) are the 5′ and 3′sequences which flank the coding region and are not translated into amino acids.

An antisense oligonucleotide can be, for example, about 5, 10, 15, 20, 25, 30, 35, 40, 45 or 50 nucleotides or more in length. An antisense nucleic acid of the invention can be constructed using chemical synthesis and enzymatic ligation reactions using procedures known in the art. For example, an antisense nucleic acid (e. g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e. g, phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Another aspect of the invention pertains to vectors, typically expression vectors, containing a nucleic acid encoding a polypeptide of the invention, a fragment thereof or a fusion protein according to the invention.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce polypeptides of the invention, fragments thereof or fusion proteins according to the invention.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

One strategy to maximize recombinant protein expression in E. coli is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention can be carried out by standard DNA synthesis techniques.

In one embodiment, the expression vector is a yeast expression vector.

Alternatively, the expression vector is a baculovirus expression vector.

In yet another embodiment, a nucleic acid of the invention is expressed in mammalian cells using a mammalian expression vector. When used in mammalian cells, the expression vector's control functions are often provided by viral regulatory elements. For example, commonly used promoters are derived from polyoma, Adenovirus 2, cytomegalovirus and Simian Virus 40.

In one embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art.

Another aspect of the invention pertains to host cells into which a recombinant expression vector of the invention has been introduced.

The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

In one embodiment, the expression characteristics of an endogenous gene within a cell, cell line or microorganism may be modified by inserting a DNA regulatory element heterologous to the endogenous gene of interest into the genome of a cell, stable cell line or cloned microorganism such that the inserted regulatory element is operatively linked with the endogenous gene and controls, modulates or activates. For example, endogenous genes which are normally “transcriptionally silent”, i.e., genes which are normally not expressed, or are expressed only at very low levels in a cell line or microorganism, may be activated by inserting a regulatory element which is capable of promoting the expression of a normally expressed gene product in that cell line or microorganism. Alternatively, transcriptionally silent, endogenous genes may be activated by insertion of a promiscuous regulatory element that works across cell types. A heterologous regulatory element may be inserted into a stable cell line or cloned microorganism, such that it is operatively linked with and activates expression of endogenous genes, using techniques, such as targeted homologous recombination, which are well known to those of skill in the art.

A host cell of the invention, such as a prokaryotic or eukaryotic host cell in culture, can be used to produce a polypeptide of the invention. Accordingly, the invention further provides methods for producing a polypeptide of the invention, a fragment thereof or a fusion protein according to the invention using the host cells of the invention. In one embodiment, the method comprises culturing the host cell of invention (into which a recombinant expression vector encoding the polypeptide has been introduced) in a suitable medium such that the polypeptide is produced. In one embodiment, the method further comprises isolating the polypeptide from the medium or the host cell.

The present invention also relates to a method for producing a recombinant host cell expressing an polypeptide according to the invention, said method comprising the steps consisting of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said polypeptide. Such recombinant host cells can be used for the production of polypeptides according to the present invention, as previously described.

The invention further relates to a method of producing a polypeptide according to the invention, which method comprises the steps consisting of: (i) culturing a transformed host cell according to the invention under conditions suitable to allow expression of said polypeptide; and (ii) recovering the expressed polypeptide.

The host cells of the invention can also be used to produce non human transgenic animals. For example, in one embodiment, a host cell of the invention is a fertilized oocyte or an embryonic stem cell into which a sequence encoding a polypeptide of the invention has been introduced. Such host cells can then be used to create non-human transgenic animals in which exogenous sequences encoding a polypeptide of the invention have been introduced into their genome or homologous recombinant animals in which endogenous encoding a polypeptide of the invention sequences have been altered. Such animals are useful for studying the function and/or activity of the polypeptide and for identifying and/or evaluating modulators of polypeptide activity.

As used herein, a “transgenic animal” is a non-human animal, typically a mammal, more typically a rodent such as a rat or mouse, in which one or more of the cells of the animal includes a transgene. Examples of transgenic animals include rodents such as mouse or rat, non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal.

A transgenic animal of the invention can be created by introducing nucleic acid encoding a polypeptide of the invention into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

In one embodiment, transgenic non-human animals can be produced which contain selected systems which allow for regulated expression of the transgene. One example of such a system is the cre/toxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

The present invention also relates to a cell vesicle which comprises a polypeptide or a nucleic acid of the invention. In one embodiment, the cell vesicle is an exosome. Within the context of this invention, the term “exosome” refers to externally released vesicles originating from the endosomic compartment or cells. More specifically, such vesicles are of endosomal origin and are secreted in the extracellular milieu following fusion of late endosomal multivesicular bodies with the plasma membrane.

The present invention also relates to an antibodies specific for an isolated polypeptide of the invention or for a fragment thereof.

The term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments.

In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of polypeptide of the invention (or a fragment thereof). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant polypeptide of the invention (or a fragment thereof) may be provided by expression with recombinant cell lines. Polypeptide of the invention (may be provided in the form of human cells expressing at their surface. Recombinant forms of polypeptide of the invention (or a fragment thereof) may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

In one embodiment the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3 A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., J. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In one embodiment, the antibody is specific for an amino acid sequence ranging from the amino acid residue at position 86 to the amino acid residue at position 189 in SEQ ID NO:1.

In one embodiment the antibody is specific for an amino acid sequence ranging from the amino acid residue at position 86 to the amino acid residue at position 174 in SEQ ID NO:2.

In one embodiment, the antibody is specific for an amino acid sequence ranging from the amino acid residue at position 86 to the amino acid residue at position 175 in SEQ ID NO:4.

In another embodiment, the invention relates to an aptamer directed against a polypeptide of the invention, a fragment thereof or a fusion protein according to the invention. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. 1997. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consist of conformationally constrained antibody variable regions displayed by a platform protein, such as E. coli Thioredoxin A, that are selected from combinatorial libraries by two hybrid methods (Colas et al., 1996).

In one embodiment, the aptamer is specific for an amino acid sequence ranging from the amino acid residue at position 86 to the amino acid residue at position 189 in SEQ ID NO:1.

In one embodiment the aptamer is specific for an amino acid sequence ranging from the amino acid residue at position 86 to the amino acid residue at position 174 in SEQ ID NO:2.

In one embodiment, the aptamer is specific for an amino acid sequence ranging from the amino acid residue at position 86 to the amino acid residue at position 175 in SEQ ID NO:4.

The present invention also relates to an immunoconjugate which consists of an antibody conjugated to a polypeptide of the invention, a fragment thereof or a fusion protein according to the invention.

Typically, the antibody may be directed against any antigen. The antigen refers to the compound any macromolecule but is typically a polypeptide. The antigen can be a part of a cell such as a cell bearing the antigen, or a microorganism e.g., bacterium, fungus, protozoan, or virus. A wide variety of proteins may be considered as antigens. Such proteins include, for example, immunoglobulins, cytokines, enzymes, hormones, cancer antigens, nutritional markers, tissue specific antigens, etc.

Alternatively, the antibody according to the invention may be directed against a cancer antigen. Known cancer antigens include, without limitation, c-erbB-2 (erbB-2 is also known as c-neu or HER-2), which is particularly associated with breast, ovarian, and colon tumor cells, as well as neuroblastoma, lung cancer, thyroid cancer, pancreatic cancer, prostate cancer, renal cancer and cancers of the digestive tract. Another class of cancer antigens is oncofetal proteins of nonenzymatic function. These antigens are found in a variety of neoplasms, and are often referred to as “tumor-associated antigens.” Carcinoembryonic antigen (CEA), and □-fetoprotein (AFP) are two examples of such cancer antigens. AFP levels rise in patients with hepatocellular carcinoma: 69% of patients with liver cancer express high levels of AFP in their serum. CEA is a serum glycoprotein of 200 kDa found in adenocarcinoma of colon, as well as cancers of the lung and genitourinary tract. Yet another class of cancer antigens is those antigens unique to a particular tumor, referred to sometimes as “tumor specific antigens,” such as heat shock proteins (e.g., hsp70 or hsp90 proteins) from a particular type of tumor. These molecules are expressed on many types of tumors, but not normally on healthy cells. Additional specific examples of cancer antigens include epithelial cell adhesion molecule (Ep-CAM/TACSTD1), mesothelin, tumor-associated glycoprotein 72 (TAG-72), gp100, Melan-A, MART-1, KDR, RCAS1, MDA7, cancer-associated viral vaccines (e.g., human papillomavirus antigens), prostate specific antigen (PSA, PSMA), RAGE (renal antigen), CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), cancer-associated ganglioside antigens, tyrosinase, gp75, C-myc, Mart1, MelanA, MUM-1, MUM-2, MUM-3, HLA-B7, Ep-CAM, tumor-derived heat shock proteins, and the like (see also, e.g., Acres et al., Curr Opin Mol Ther 2004 February, 6:40-7; Taylor-Papadimitriou et al., Biochim Biophys Acta. 1999 Oct. 8; 1455(2-3):301-13; Emens et al., Cancer Biol Ther. 2003 July-August; 2(4 Suppl 1):S161-8; and Ohshima et al., Int J Cancer. 2001 Jul. 1; 93(1):91-6). Other exemplary cancer antigen targets include CA 195 tumor-associated antigen-like antigen (see, e.g., U.S. Pat. No. 5,324,822) and female urine squamous cell carcinoma-like antigens (see, e.g., U.S. Pat. No. 5,306,811), and the breast cell cancer antigens described in U.S. Pat. No. 4,960,716.

The antibody according to the invention may also target protein antigens, carbohydrate antigens, or glycosylated proteins. For example, the antibody can target glycosylation groups of antigens that are preferentially produced by transformed (neoplastic or cancerous) cells, infected cells, and the like (cells associated with other immune system-related disorders). In one aspect, the antigen is a tumor-associated antigen. In an exemplary aspect, the antigen is O-acetylated-GD2 or glypican-3. In another particular aspect, the antigen is one of the Thomsen-Friedenreich (TF) antigens (TFAs).

The antibody according to the invention can also exhibit specificity for a cancer-associated protein. Such proteins can include any protein associated with cancer progression. Examples of such proteins include angiogenesis factors associated with tumor growth, such as vascular endothelial growth factors (VEGFs), fibroblast growth factors (FGFs), tissue factor (TF), epidermal growth factors (EGFs), and receptors thereof; factors associated with tumor invasiveness; and other receptors associated with cancer progression (e.g., one of the HER1-HER4 receptors).

Alternatively the antibody according to the invention can be specific for a virus, a bacteria or parasite associated target. For example, the antibody may be specific for a virus-associated target such as an HIV protein (e.g., gp120 or gp41), CMV or other viruses, such as hepatitis C virus (HCV).

Techniques for conjugating molecule to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

The polypeptides, nucleic acid molecules, vectors, host cells, antibodies; aptamers and immunoconjugates of the invention may be particularly suitable for therapeutic purposes.

In some embodiments, the polypeptides (e.g. MICA-B1 and MICA-B2 polypetides), fusion proteins and immunoconjugates which are NKG2D agonists could be suitable for activating NK cells. Said compounds are thus suitable for the treatment of cancer and infectious diseases.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Infectious diseases that can be treated by the polypeptides (e.g. MICA-B1 and MICA-B2 polypetides), fusion proteins and immunoconjugates which are NKG2D agonists are caused by infectious agents including, but not limited to, viruses, bacteria, fungi, protozoa and parasites. Bacterial diseases that can be treated or prevented by the polypeptides (e.g. MICA-B1 and MICA-B2 polypetides), fusion proteins and immunoconjugates which are NKG2D agonists are caused by bacteria including, but not limited to, mycobacteria rickettsia, mycoplasma, neisseria and legionella. Protozoal diseases that can be the polypeptides (e.g. MICA-B1 and MICA-B2 polypetides), fusion proteins and immunoconjugates which are NKG2D agonists are caused by protozoa including, but not limited to, leishmania, kokzidioa, and Trypanosoma. Parasitic diseases that can be the polypeptides (e.g. MICA-B1 and MICA-B2 polypetides), fusion proteins and immunoconjugates which are NKG2D agonists are caused by parasites including, but not limited to, chlamydia and rickettsia. Viral diseases that can be the polypeptides (e.g. MICA-B1 and MICA-B2 polypetides), fusion proteins and immunoconjugates which are NKG2D agonists include, but are not limited to, those caused by hepatitis type A, hepatitis type B, hepatitis type C, influenza, varicella, adenovirus, herpes simplex type I (HSV-I), herpes simplex type II (HSV-II), rinderpest, rhino virus, echo virus, rotavirus, respiratory syncytial virus, papilloma virus, papova virus, cytomegalovirus, echinovirus, arbovirus, huntavirus, coxsachie virus, mumps virus, measles virus, rubella virus, polio virus, human immunodeficiency virus type I (HIV-I), and human immunodeficiency virus type II (HIV-II). Cancers that can be the polypeptides (e.g. MICA-B1 and MICA-B2 polypetides), fusion proteins and immunoconjugates which are NKG2D agonists include, but are not limited to human sarcomas and carcinomas, e.g., fibrosarcoma, myxosarcoma, liposarcoma, chondrosarcoma, osteogenic sarcoma, carcinoma of the head/neck, chordoma, angio sarcoma, endothelio sarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms1 tumor, cervical cancer, testicular tumor, lung carcinoma, small cell lung carcinoma, bladder carcinoma, epithelial carcinoma, glioma, glioblastoma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, and heavy chain disease.

In some embodiments, the polypeptides (e.g. MICA-D polypetides), fusion proteins, antibodies (e.g. antibodies against MICA-B1 or MICA-B2 polypeptides) and immunoconjugates which are NKG2D antagonists could be suitable for blocking the activation of NK cells. Said compounds are thus suitable for the treatment of autoimmune diseases and inflammatory conditions.

As used herein, an “autoimmune disease” is a disease or disorder arising from and directed at an individual's own tissues. Examples of autoimmune diseases include, but are not limited to Addison's Disease, Allergy, Alopecia Areata, Alzheimer's disease, Antineutrophil cytoplasmic antibodies (ANCA)-associated vasculitis, Ankylosing Spondylitis, Antiphospholipid Syndrome (Hughes Syndrome), arthritis, Asthma, Atherosclerosis, Atherosclerotic plaque, autoimmune disease (e.g., lupus, RA, MS, Graves' disease, etc.), Autoimmune Hemolytic Anemia, Autoimmune Hepatitis, Autoimmune inner ear disease, Autoimmune Lymphoproliferative syndrome, Autoimmune Myocarditis, Autoimmune Oophoritis, Autoimmune Orchitis, Azoospermia, Behcet's Disease, Berger's Disease, Bullous Pemphigoid, Cardiomyopathy, Cardiovascular disease, Celiac Sprue/Coeliac disease, Chronic Fatigue Immune Dysfunction Syndrome (CFIDS), Chronic idiopathic polyneuritis, Chronic Inflammatory Demyelinating, Polyradicalneuropathy (CIPD), Chronic relapsing polyneuropathy (Guillain-Barré syndrome), Churg-Strauss Syndrome (CSS), Cicatricial Pemphigoid, Cold Agglutinin Disease (CAD), chronic obstructive pulmonary disease (COPD), CREST syndrome, Crohn's disease, Dermatitis, Herpetiformus, Dermatomyositis, diabetes, Discoid Lupus, Eczema, Epidermolysis bullosa acquisita, Essential Mixed Cryoglobulinemia, Evan's Syndrome, Exopthalmos, Fibromyalgia, Goodpasture's Syndrome, Hashimoto's Thyroiditis, Idiopathic Pulmonary Fibrosis, Idiopathic Thrombocytopenia Purpura (ITP), IgA Nephropathy, immunoproliferative disease or disorder (e.g., psoriasis), Inflammatory bowel disease (IBD), including Crohn's disease and ulcerative colitis, Insulin Dependent Diabetes Mellitus (IDDM), Interstitial lung disease, juvenile diabetes, Juvenile Arthritis, juvenile idiopathic arthritis (JIA), Kawasaki's Disease, Lambert-Eaton Myasthenic Syndrome, Lichen Planus, lupus, Lupus Nephritis, Lymphoscytic Lypophisitis, Meniere's Disease, Miller Fish Syndrome/acute disseminated encephalomyeloradiculopathy, Mixed Connective Tissue Disease, Multiple Sclerosis (MS), muscular rheumatism, Myalgic encephalomyelitis (ME), Myasthenia Gravis, Ocular Inflammation, Pemphigus Foliaceus, Pemphigus Vulgaris, Pernicious Anaemia, Polyarteritis Nodosa, Polychondritis, Polyglandular Syndromes (Whitaker's syndrome), Polymyalgia Rheumatica, Polymyositis, Primary Agammaglobulinemia, Primary Biliary Cirrhosis/Autoimmune cholangiopathy, Psoriasis, Psoriatic arthritis, Raynaud's Phenomenon, Reiter's Syndrome/Reactive arthritis, Restenosis, Rheumatic Fever, rheumatic disease, Rheumatoid Arthritis, Sarcoidosis, Schmidt's syndrome, Scleroderma, Sjörgen's Syndrome, Stiff-Man Syndrome, Systemic Lupus Erythematosus (SLE), systemic scleroderma, Takayasu Arteritis, Temporal Arteritis/Giant Cell Arteritis, Thyroiditis, Type 1 diabetes, Type 2 diabetes, Ulcerative colitis, Uveitis, Vasculitis, Vitiligo, and Wegener's Granulomatosis.

The term “inflammatory condition” as used herein refers to acute or chronic localized or systemic responses to harmful stimuli, such as pathogens, damaged cells, physical injury or irritants, that are mediated in part by the activity of cytokines, chemokines, or inflammatory cells (e.g., neutrophils, monocytes, lymphocytes, macrophages) and is characterized in most instances by pain, redness, swelling, and impairment of tissue function. The inflammatory condition may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graftversus-host disease, transplant rejection, sickle cell anemia, nephrotic syndrome, toxicity of agents such as OKT3, cytokine therapy, and cirrhosis.

A further aspect of the invention relates to a pharmaceutical composition comprising an amount of the polypeptides, nucleic acid molecules, vectors, host cells, antibodies; aptamers and immunoconjugates of the invention. Indeed, the polypeptides, nucleic acid molecules, vectors, host cells, antibodies; aptamers and immunoconjugates of the invention of the invention may be combined with pharmaceutically acceptable excipients, and optionally sustained-release matrices, such as biodegradable polymers, to form pharmaceutical compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The polypeptides, nucleic acid molecules, vectors, host cells, antibodies; aptamers and immunoconjugates of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The polypeptides, nucleic acid molecules, vectors, host cells, antibodies; aptamers and immunoconjugates of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the polypeptides formulated for parenteral administration, such as intravenous or intramuscular injection, other pharmaceutically acceptable forms include, e.g. tablets or other solids for oral administration; liposomal formulations; time release capsules; and any other form currently used.

The present invention also pertains to diagnostic assays, prognostic assays, and monitoring assays. In particular, one aspect of the present invention relates to diagnostic assays for determining expression of a polypeptide or nucleic acid of the invention and/or activity of a polypeptide of the invention, in the context of a biological sample (e.g., blood, serum, cells, tissue) to thereby determine whether an individual is afflicted with a disease or disorder, or is at risk of developing a disorder, associated with aberrant expression or activity of a polypeptide of the invention.

The invention also provides for prognostic (or predictive) assays for determining whether an individual is at risk of developing a disorder associated with aberrant expression or activity of a polypeptide of the invention. Such assays can be used for prognostic or predictive purpose to thereby prophylactically treat an individual prior to the onset of a disorder characterized by or associated with aberrant expression or activity of a polypeptide of the invention.

Yet another aspect of the invention pertains to monitoring the influence of agents (e.g., drugs or other compounds) on the expression or activity of a polypeptide of the invention, in clinical trials or treatments.

An exemplary method for detecting the presence or absence of a polypeptide or nucleic acid of the invention in a biological sample involves obtaining a biological sample from a test subject and contacting the biological sample with a compound or an agent capable of detecting a polypeptide or nucleic acid (e.g., mRNA, genomic DNA) of the invention such that the presence of a polypeptide or nucleic acid of the invention is detected in the biological sample. A preferred agent for detecting mRNA or genomic DNA encoding a polypeptide of the invention is a labeled nucleic acid probe capable of hybridizing to mRNA or genomic DNA encoding a polypeptide of the invention. The nucleic acid probe can be, for example, a full-length cDNA, such as the nucleic acid of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D), or a portion thereof, such as an oligonucleotide of at least 15, 30, 50, 100, 250 or 500 nucleotides in length and sufficient to specifically hybridize under stringent conditions to a mRNA or genomic DNA encoding a polypeptide of the invention. Other suitable probes for use in the diagnostic assays of the invention are described herein.

A preferred agent for detecting a polypeptide of the invention is an antibody capable of binding to a polypeptide of the invention, typically an antibody with a detectable label. Antibodies may be prepared according to the methods as above describes.

The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells and biological fluids isolated from a subject, as well as tissues, cells and fluids present within a subject.

The detection method of the invention can be used to detect mRNA, protein, or genomic DNA in a biological sample in vitro as well as in vivo.

Methods for determining a quantity of mRNA are well known in the art. For example nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The thus extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of Amplification include ligase chain reaction (LCR), transcription-mediated amplification (TMA), strand displacement amplification (SDA) and nucleic acid sequence based amplification (NASBA), quantitative new generation sequencing of RNA (NGS).

Nucleic acids (polynucleotides) comprising at least 10 nucleotides and exhibiting sequence complementarity or homology to the mRNA of interest herein find utility as hybridization probes or amplification primers. It is understood that such nucleic acids need not be completely identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin).

Probes typically comprise single-stranded nucleic acids of between 10 to 1000 nucleotides in length, for instance of between 10 and 800, more preferably of between 15 and 700, typically of between 20 and 500 nucleotides. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

Nucleic acids which may be used as primers or probes in the above amplification and detection method may be assembled as a kit. Such a kit includes consensus primers and molecular probes. A preferred kit also includes the components necessary to determine if amplification has occurred. A kit may also include, for example, PCR buffers and enzymes; positive control sequences, reaction control primers; and instructions for amplifying and detecting the specific sequences.

In one embodiment, the methods of the invention comprise the steps of providing total RNAs extracted from cells and subjecting the RNAs to amplification and hybridization to specific probes, more particularly by means of a quantitative or semi-quantitative RT-PCR.

Probes made using the disclosed methods can be used for nucleic acid detection, such as in situ hybridization (ISH) procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pinkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. J. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publications Nos. 2006/0246524; 2006/0246523, and 2007/0117153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In one embodiment, the methods of the invention comprise the steps of providing total RNAs extracted from cells and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

The expression level of a gene may be expressed as absolute expression level or normalized expression level. Both types of values may be used in the present method. The expression level of a gene is preferably expressed as normalized expression level when quantitative PCR is used as method of assessment of the expression level because small differences at the beginning of an experiment could provide huge differences after a number of cycles.

Typically, expression levels are normalized by correcting the absolute expression level of a gene by comparing its expression to the expression of a gene that is not relevant for determining the cancer stage of the patient, e.g., a housekeeping gene that is constitutively expressed. Suitable genes for normalization include housekeeping genes such as the actin gene ACTB, ribosomal 18S gene, GUSB, PGK1 and TFRC. This normalization allows comparing the expression level of one sample, e.g., a patient sample, with the expression level of another sample, or comparing samples from different sources.

In vitro techniques for detection of a polypeptide of the invention include enzyme linked immunosorbent assays (ELISAs), Western blots, immunoprecipitations and immunofluorescence. In vitro techniques for detection of genomic DNA include Southern hybridizations, DNA arrays, exome arrays, SNP arrays, HST sequencing. Furthermore, in vivo techniques for detection of a polypeptide of the invention include introducing into a subject a labeled antibody directed against the polypeptide. For example, the antibody can be labeled with a radioactive marker whose presence and location in a subject can be detected by standard imaging techniques.

The methods of the invention can also be used to detect genetic lesions or mutations in a gene of the invention, thereby determining if a subject with the lesioned gene is at risk for a disorder characterized aberrant expression or activity of a polypeptide of the invention. In preferred embodiments, the methods include detecting, in a sample of cells from the subject, the presence or absence of a genetic lesion or mutation characterized by at least one of an alteration affecting the integrity of a gene encoding the polypeptide of the invention, or the mis-expression of the gene encoding the polypeptide of the invention. For example, such genetic lesions or mutations can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of a the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

In an alternative embodiment, mutations in a selected gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA.

In yet another embodiment, any of a variety of sequencing reactions known in the art can be used to directly sequence the selected gene and detect mutations by comparing the sequence of the sample nucleic acids with the corresponding wild-type (control) sequence. The one skilled in the art is familiar with several methods for sequencing of polynucleotides. These include, but are not limited to, Sanger sequencing (also referred to as dideoxy sequencing) and various sequencing-by-synthesis (SBS) methods as reviewed by Metzger (Metzger ML 2005, Genome Research 1767), sequencing by hybridization, by ligation (for example, WO 2005/021786), by degradation (for example, U.S. Pat. Nos. 5,622,824 and 6,140,053), nanopore sequencing. Preferably in a multiplex assay deep high throughput sequencing is preferred. The term “deep high throughput sequencing” refers to a method of sequencing a plurality of nucleic acids in parallel. See e.g., Bentley et al, Nature 2008, 456:53-59. The leading commercially available platforms produced by Roche/454 (Margulies et al., 2005a), Illumina/Solexa (Bentley et al., 2008), Life/APG (SOLiD) (McKernan et al., 2009), Ion Torrent PGM/Proton ( ) and Pacific Biosciences (Eid et al., 2009) may be used for deep HT sequencing. For example, in the 454 method, the DNA to be sequenced is either fractionated and supplied with adaptors or segments of DNA can be PCR-amplified using primers containing the adaptors. The adaptors are nucleotide 25-mers required for binding to the DNA Capture Beads and for annealing the emulsion PCR Amplification Primers and the Sequencing Primer. The DNA fragments are made single stranded and are attached to DNA capture beads in a manner that allows only one DNA fragment to be attached to one bead. Next, the DNA containing beads are emulsified in a water-in-oil mixture resulting in microreactors containing just one bead. Within the microreactor, the fragment is PCR-amplified, resulting in a copy number of several million per bead. After PCR, the emulsion is broken and the beads are loaded onto a pico titer plate. Each well of the pico-titer plate can contain only one bead. Sequencing enzymes are added to the wells and nucleotides are flowed across the wells in a fixed order. The incorporation of a nucleotide results in the release of a pyrophosphate, which catalyzes a reaction leading to a chemiluminescent signal. This signal is recorded by a CCD camera and a software is used to translate the signals into a DNA sequence. In the lllumina method (Bentley (2008)), single stranded, adaptor-supplied fragments are attached to an optically transparent surface and subjected to “bridge amplification”. This procedure results in several million clusters, each containing copies of a unique DNA fragment. DNA polymerase, primers and four labeled reversible terminator nucleotides are added and the surface is imaged by laser fluorescence to determine the location and nature of the labels. Protecting groups are then removed and the process is repeated for several cycles. The SOLiD process (Shendure (2005)) is similar to 454 sequencing, DNA fragments are amplified on the surface of beads. Sequencing involves cycles of ligation and detection of labeled probes. The Ion Torrent sequencers uses a post-light, semiconductor-based technology that dramatically reduce the sequencing cost. Several other techniques for high-throughput sequencing are currently being developed. Examples of such are The Helicos system (Harris (2008)), Complete Genomics (Drmanac (2010)) and Pacific Biosciences (Lundquist (2008)). For example, a PACIFIC BIOSCIENCES' SMRT™ (Single Molecule Real Time sequencing) sequencing platform may be used. Said technology uses a real time sequencing by synthesis and can produce reads of up to 1000 by in length as a result of not being limited by reversible terminators. Raw read throughput that is equivalent to one-fold coverage of a diploid human genome can be produced per day using this technology. As this is an extremely rapidly developing technical field, the applicability to the present invention of high throughput sequencing methods will be obvious to a person skilled in the art.

In other embodiments, alterations in electrophoretic mobility will be used to identify mutations in genes. For example, single strand conformation polymorphism (SSCP) may be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a 'GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6: 1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

In one embodiment, the methods of the invention comprise the step consisting of detecting a SNP selected from the group consisting of Rs41558312 (A/G), Rs41556715 (A-G), Rs1051792 (A/G), Rs199503730 (-G), Rs61738275 (C/T), Rs41558418 (-G), Rs41553217 (A/G).

The methods of the invention can also comprise the step consisting of detecting one or more autoantibodies recognizing one or more polypeptide of the invention selected from the group consisting of SEQ ID NO:1 (MICA-A), SEQ ID NO:2 (MICA-B1), SEQ ID NO:3 (MICA-B2); SEQ ID NO:4 (MICA-C), SEQ ID NO:5 (MICA-D) and variants thereof.

The term “autoantibody”, as used herein, has meaning accepted in the art, and refers to an antibody that is produced by the immune system of a subject and that is directed against subject's own proteins. For example, autoantibodies may attack the body's own cells, tissues, and/or organs, causing inflammation and damage. In one embodiment, detection of aid autoantibodies may be particularly suitable for determining whether a subject is at risk of graft rejection.

Typically, the methods of the present invention can involve detection of a biomarker-antigen complex formed between the protein biomarker (i.e. a polypeptide of the invention) and an autoantibody present in the biological sample tested. In the practice of the invention, detection of such a complex may be performed by any suitable method (see, for example, E. Harlow and A. Lane, “Antibodies: A Laboratories Manual”, 1988, Cold Spring Harbor Laboratory: Cold Spring Harbor, N.Y.). For example, detection of a biomarker-antibody complex may be performed using an immunoassay. A wide range of immunoassay techniques is available, including radioimmunoassay, enzyme immunoassays (EIA), enzyme-linked immunosorbent assays (ELISA), and immunofluorescence immunoprecipitation. Immunoassays are well known in the art. Methods for carrying out such assays as well as practical applications and procedures are summarized in textbooks. Examples of such textbooks include P. Tijssen, In: Practice and theory of enzyme immunoassays, eds. R. H. Burdon and v. P. H. Knippenberg, Elsevier, Amsterdam (1990), pp. 221-278 and various volumes of Methods in Enzymology, Eds. S. P. Colowick et al., Academic Press, dealing with immunological detection methods, especially volumes 70, 73, 74, 84, 92 and 121. Immunoassays may be competitive or non-competitive.

For example, any of a number of variations of the sandwich assay technique may be used to perform an immunoassay. Briefly, in a typical sandwich assay applied to the detection of, for example, autoantibodies according to the present invention, an unlabeled polypeptide of the invention or fragment thereof is immobilized on a solid surface (as described above) and the biological sample to be tested is brought into contact with the bound biomarker for a time and under conditions allowing formation of a biomarker-antibody complex. Following incubation, an antibody that is labeled with a detectable moiety and that specifically recognizes antibodies from the species tested (e.g., an anti-human IgG for human subjects) is added and incubated under conditions allowing the formation of a ternary complex between any biomarker-bound autoantibody and the labeled antibody. Any unbound material is washed away, and the presence of any autoantibody in the sample is determined by observation/detection of the signal directly or indirectly produced by the detectable moiety. Variations on this assay include an assay, in which both the biological sample and the labeled antibody are added simultaneously to the immobilized polypeptide biomarker. The second antibody (i.e., the antibody added in a sandwich assay as described above) may be labeled with any detectable moiety, i.e., any entity which, by its chemical nature, provides an analytically identifiable signal allowing detection of the ternary complex, and consequently detection of the biomarker-antibody complex.

Detection may be either qualitative or quantitative. Methods for labeling biological molecules such as antibodies are well-known in the art (see, for example, “Affinity Techniques. Enzyme Purification: Part B”, Methods in Enzymol., 1974, Vol. 34, W. B. Jakoby and M. Wilneck (Eds.), Academic Press: New York, N.Y.; and M. Wilchek and E. A. Bayer, Anal. Biochem., 1988, 171: 1-32).

The most commonly used detectable moieties in immunoassays are enzymes and fluorophores. In the case of an enzyme immunoassay (EIA or ELISA), an enzyme such as horseradish perodixase, glucose oxidase, beta-galactosidase, alkaline phosphatase, and the like, is conjugated to the second antibody, generally by means of glutaraldehyde or periodate. The substrates to be used with the specific enzymes are generally chosen for the production of a detectable color change, upon hydrolysis of the corresponding enzyme. In the case of immunofluorescence, the second antibody is chemically coupled to a fluorescent moiety without alteration of its binding capacity. After binding of the fluorescently labeled antibody to the biomarker-antibody complex and removal of any unbound material, the fluorescent signal generated by the fluorescent moiety is detected, and optionally quantified. Alternatively, the second antibody may be labeled with a radioisotope, a chemiluminescent moiety, or a bioluminescent moiety.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a gene encoding a polypeptide of the invention

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of a polypeptide of the invention (e.g. retinal degenerative diseases). The kit, for example, can comprise a labeled compound or agent capable of detecting the polypeptide or mRNA encoding the polypeptide in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for observing that the tested subject is suffering from or is at risk of developing a disorder associated with aberrant expression of the polypeptide if the amount of the polypeptide or mRNA encoding the polypeptide is above or below a normal level.

The kit can comprise, for example: (1) a first antibody (e.g., attached to a solid support) which binds to a polypeptide of the invention; and, optionally, (2) a second, different antibody which binds to either the polypeptide or the first antibody and is conjugated to a detectable agent.

The kit can comprise, for example: (1) an oligonucleotide, e.g., a detectably labeled oligonucleotide, which hybridizes to a nucleic acid sequence encoding a polypeptide of the invention or (2) a pair of primers useful for amplifying a nucleic acid molecule encoding a polypeptide of the invention.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

In one embodiment, the kit comprises the oligonucleotide primers SEQ ID NO:16 and SEQ ID NO:17 for amplifying the nucleic acid molecule consisting of SEQ ID NO:6 (MICA-A).

In one embodiment, the kit comprises the oligonucleotide primers SEQ ID NO:18 and SEQ ID NO:19 for amplifying the nucleic acid molecule consisting of SEQ ID NO:7 (MICA-B1).

In one embodiment, the kit comprises the oligonucleotide primers SEQ ID NO:20 and SEQ ID NO:21 for amplifying the nucleic acid molecule consisting of SEQ ID NO:8 (MICA-B2).

In one embodiment, the kit comprises the oligonucleotide primers SEQ ID NO:22 and SEQ ID NO:23 for amplifying the nucleic acid molecule consisting of SEQ ID NO:9 (MICA-C).

In one embodiment, the kit comprises the oligonucleotide primers SEQ ID NO:24 and SEQ ID NO:25 for amplifying the nucleic acid molecule consisting of SEQ ID NO:10 (MICA-D).

The invention will be further illustrated by the following figures and examples. However, these examples and figures should not be interpreted in any way as limiting the scope of the present invention.

### FIGURES

FIG. 1. Detection of MICA alternative transcripts in cultured human endothelial cells EC) bearing the MICA*010/*017 haplotype by PCR. RNA was purified from two MICA heterozygous cultures (MICA*002/*009 and MOCA *010/*017). RT-PCR were performed using primers encompassing the full length MICA cDNA (1264 bp). PCR product were separated on agarose gel and stained with ethidium bromide.

FIG. 2. Schematic representations of MICA gene (a), wild type (WT) full MICA mRNA (b), the five novel MICA alternative transcripts (c) and the respective predicted proteins (MICA-WT, MICA-A, MICA-B1, MICA-B2, MICA-C; MICA-D)(d)

FIG. 3 shows a schematic representation of MICA alleles found associated (grey boxes) with MICA isoforms among the panel of MICA alleles tested (n=22).

FIG. 4: MICA *015 and *017 alleles associate with a <<G>> deletion at the 5′ end donor splice site of MICA intron 4.

FIG. 5: Detection of MICA alternative transcripts in cells bearing the MICA *015 or *017 alleles by PCR. RNAs were purified from two MICA homozygous cell lines (OMW *015/*015 and WIN *017/*017) and two heterozygous EC cultures (*010/*017 and *018/*017). Two irrelevant EC cultures were used as negative controls. RT-PCR were performed using primers encompassing the full length MICA cDNA (1264 bp). PCR products were separated on agarose gel and stained with ethidium bromide. SM: size marker.

FIG. 6: Detection of MICA alternative transcripts in cells bearing the MICA*015 or *017 allele by PCR using isoform-specific designed primer pairs. RNAs were purified from an heterozygous EC cultures (*010/*017), two MICA homozygous cell lines (OMW *015/*015 and WIN *017/*017) and peripheral blood leucocytes (PBLs) from two heterozygous donors. RT-PCR were performed using isoform-specific primers and PCR products were separated on agarose gel containing ethidium bromide.

FIG. 7: Expression of MICA alternative transcripts (a) and isoforms in COS (b) transfected cells. COS cells were transfected with plasmids containing full length cDNA s for MICA WT or isoforms A, B1, B2, C and D in frame with a Flag peptide (M2) in 3′. After transfection (48 h), cells were used for RNA isolation or lyzed for protein analysis. (a) RT-PCR were performed using isoform-specific (upper panel) and HPRT (lower panel) primers and PCR products were separated on agarose gel containing ethidium bromide. (b) Cells lysates were treated ON with (+) or without (−) PNGaseF according to manufacturer's recommendations. Cell lysates were separated by 12% SDS-PAGE, Proteins were immunoblotted with an anti-FLAG monoclonal antibody.

FIG. 8: Cellular localisation of MICA isoform expression analyzed by confocal microscopy on COS-7 transfected cells using an anti-Flag (M2) monoclonal antibody. Flag intracellular detection was achieved 48 h post-transfection after cell fixation and permeabilization. COS cells transfected with the WT cDNA for MICA are used as posistive controls. Immunofluorescence staining was obtained using an FITC-labeled secondary antibody and shown in black and white, specific staining appears in white.

FIG. 9: MICA-B1 and MICA-B2 isoforms are detected by anti-MICA specific antibodies in transfected cells. Detection of MICA-B1 in cell lysates from 293 HEK transfected cells. HEK cells were transfected with plasmids containing full length cDNA s for MICA WT or isoforms A, B1, B2, C and D in frame with a Flag peptide (M2) in 3′. After transfection (48 h), cells were lyzed for protein analysis. Cell lysates were separated by SDS-PAGE, Proteins were immunoblotted with an anti-FLAG monoclonal antibody (upper panel), anti-MICA (AMO1, medium panel) or anti-GAPDH (lower panel) antibodies. Size markers are indicated (kDa).

FIG. 10. A). MICA-B2 is a novel ligand for the NKG2D receptor. COS cells were transfected with cDNA for WT MICA or MICA isoforms. Forty-eight hours post-transfection cells welle incubated with recombinant NKG2D-Fc chimeric protein. Binding of NKG2D was revealed using an anti-IgG labeled to FITC. Staining was analyzed by fluorescent microscopy and positive signal was converted into black and white picture, positive cells intercating with NKG2D-Fc are shown in black. COS cells transfected with the WT cDNA for MICA are used as positive controls. B) Visualisation of NKG2D binding on MICA-B2 isoform expression analyzed by confocal microscopy on COS-7 transfected cells using a recombinant NKG2D-Fc protein. Transfected cells were detected using an anti-Flag (M2) monoclonal antibody. NKG2D-Fc binding and Flag intracellular detection were achieved 48 h post-transfection before and after cell fixation and permeabilization, respectively. COS cells transfected with the WT cDNA for MICA are used as positive controls. Immunofluorescence staining was obtained using an FITC-labeled secondary antibody and shown in black and white, specific staining appears in white.

FIG. 11: Regulation of NKG2D mediated by MICA isoforms on CHO transfectants. NKL cells were incubated overnight with CHO cells stably transfected with MICA WT (*002), MICA-B1, MICA-B2 or mock transfected (TNKL+CHO). After incubation, NKL cells were harvested and immunostained for NKG2D using a specific antibody. Cells were then incubated with a phycoerythrin-labelled secondary antibody and fixed in paraformaldehyde. Fluorecence was analyzed on 10,000 cells using a Facs CANTO. Results are expressed as percentages calculated as a ratio of basal NKG2D level on NK cells (100% correspond to the basal level of NKG2D on NKL cells not incubated with CHO transfectants). Histograms are representative of 3 independent experiments.

FIG. 12. Soluble recombinant isoform MICA-B2 downregulates NKG2D expression on NK cells. NKL cells were incubated overnight without or with soluble recombinant MICA-B2 at the indicated concentrations. After incubation, NKL cells were harvested and immunostained for NKG2D using a specific antibody. Cells were then incubated with a phycoerythrin-labelled secondary antibody and fixed in paraformaldehyde. Fluorecence was analyzed on 10,000 cells using a Facs CANTO. Histograms are representative of 3 independent experiments. Intensity of fluorescence (Geo means) are indicated. Percentages of inhibition are calculated as a ratio from NKL cells without recombinant protein.

FIG. 13: NK cell activation mediated by MICA isoforms on CHO transfectants. NK cells were incubated for 4 h with CHO cells stably transfected with MICA WT (*002), MICA-B1, MICA-B2 or mock transfected (PBMC+CHO). After incubation, PBMC cells were harvested and immunostained for CD3, NKp46, CD107, IFNg using a specific antibodies. CD3 negative/NKP46 positive cells were considered as NK cells and gated for CD107a and IFNg analysis. Fluorecence was analyzed on 10,000 cells in the gate using a Facs CANTO. Results are expressed as percentages of positive cells. Histograms are representative of 3 independent experiments.

FIG. 14: Regulation of NKG2D mediated by MICA isoforms. NKL cells were incubated overnight with HEK cells transfected with MICA WT (*002), MICA-B1, MICA-B2 or MICA-D or mock transfected (TX control). After incubation, NKL cells were harvested and immunostained for NKG2D using a specific antibody. Cells were then incubated with a phycoerythrin-labelled secondary antibody and fixed in paraformaldehyde. Fluorecence was analyzed on 10,000 cells using a Facs CANTO. Histograms represents means of fluorescence intensity (MFI) and are representative of 3 independent experiments. Percentage of basal NKG2D expression are calculated as a ratio from NKL cells incubated with mock transfected HEK cells.

### EXAMPLE 1

### Material & Methods

Cloning and Expression of MICA Splicing Variants

Total RNA from primary EC cultures was extracted with TriZol and trace amounts of DNA were removed by DNase I digestion and RNA clean up steps (Life Technologies SAS, Saint Aubin, France). After reverse transcription, the full cDNAs and splice variants were amplified by PCR with Taq DNA polymerase (Invitrogen, Carlsbad, Calif., USA) with primers targeting the start andstop codon of the full length MICA sequence (PCR product: 1264 bp). The primers used were: MICA5UTR/5′->3′=GTC GGG GCC ATG GGG CT, MICA3UTR/5′->3′=TCA TAG GTC AGG AAA CTG AGG. PCR products were separated on agarose gel and extracted by phenol/chloroform method before ligation into Strataclone™ PCR cloning kit (Stratagene, Massy, France) for plasmid production, sequencing and subcloning. Cloning was achieved into pCMV-3Tag epitope tagging mammalian expression vector containing three copies of FLAG in 3′ (Stratagene). Large scale production of endotoxin-free plasmids was performed using Nucleobond™ kit (Macherey-Nalgene EURL, Hoerd, France). Plasmids were transfected in COS-7 and 293HEK cells with DEAE dextran or lipofectamine2000 according to manufacturer's recommendations (Invitrogen, Life Technologies SAS). Transfected cells were used for analyses, 48 h after transfrection.

Endothelial Cell Isolation and Cell Culture

Primary cultures of human vascular ECs (HAEC) are isolated and characterized as we previously described (Coupel et al., 2004). ECs were cultured in Endothelial Cell Basal Medium (ECBM) supplemented with 10% fetal calf serum (FCS), 0.004 mL/mL ECGS/Heparin, 0.1 ng/mL hEGF, 1 ng/mL hbFGF, 1 μg/mL hydrocortisone, 50 μg/mL gentamicin and 50 ng/mL amphotericin B (C-22010, PromoCell, Heidelberg, Germany). ECs were used between passage 2 and 5. HEK and COS-7 cells were grown in DMEM medium supplemented with 10% FCS.

Reagents and Antibodies

The following mAbs were used: anti-MICA (AMO1) and MICA/B (BAM01, BAMO3) were for BamOmab (Tubingen, Germany), anti-GAPDH (both from Chemicon, Val de Fontenay, France) anti-NKG2D mAbs as well as NKG2D-Fc protein were purchased from R&D Systems, (Lille, France), anti-CD107a (clone H4A3) and anti-IFNg were from Miltenyi biotech. FITC and PE-conjugated anti-mouse F(ab′)2 and anti-human IgG were from Jackson Immunoresearch Laboratories (West Grove, PE). For protein stability analysis, confluent EC monolayers were incubated with cycloheximide (CHX, 50 μM, Sigma-Aldrich, St Louis, Mo.) for the indicated period of time. For inhibition of soluble MICA release, ECs were treated with galardin (GM6001, 50 μg/ml, Sigma-Aldrich) or GI254023X (kindly provided by GSK) for the indicated period of time.

MICA Genotyping

MICA typing was performed from genomic DNA as we previously described (Tonnerre et al., 2010). MICA exons were amplified with the following primers: MICA1-F5′-ACGCGTTGTCTGTCCTGGAA-3′(SEQ ID NO:26), MICA1-R 5′-GAGGTGCAAAAGGGAAGATG-3′(SEQ ID NO:27) for exon1, MICA2-F 5′-ATTTCCTGCCCCAGGAAGGTTGG-3′SEQ ID NO:28) and MICA2-R 5′-AGACAGGTCCCTGCTCTCTG-3′(SEQ ID NO:29) for exon2, MICA3-F 5′-TTCGGGAATGGAGAAGTCACTGC-3′ (SEQ ID NO:29), MICA3-R 5′-AAATGCCTTCATCCATAGCACAG-3′(SEQ ID NO:30) for exon3; MICA4-F 5′-GACTTGCAGGTCAGGGGTCCC-3′ (SEQ ID NO:31), MICA4-R 5′-TGTCCCTACCCTGGCCTGACC-3′(SEQ ID NO:32) for exon 4, MICA5-F 5′-CCTTTTTTTCAGGGAAAGTGC-3′(SEQ ID NO:33), MICA5-R 5′-CCTTACCATCTCCAGAAACTGC-3′(SEQ ID NO:34) for exon5, and MICA6-F; 5′-GATGTTGATGGAGTGATGGGA-3′ (SEQ ID NO:35), MICA6-R; 5-‘ATGTTGATCAGGATGGTCTCGATC-3’(SEQ ID NO:36) for exon 6.

PCR for MICA promoter, exons 1, 5, 6 and 5′UTR were performed using 100 ng of DNA, 12.5 mM dNTPs, lx Taq buffer, 2 mM MgCl2, 0.1 U Taq DNA polymerase (Invitrogen, Carlsbad, Calif.) and 10 pM of each oligonucleotide. For MICA exons 2, 3 and 4, we first performed a PCR using 100 ng DNA, 15 pM of each primer (Katsuyama et al., 1999), 12.5 mM dNTPs, 1 U of Herculase® Taq (Stratagene, La Jolla, Calif.). Then, nested PCR were performed using 1 μL of PCR product and conditions reported above for exons 1, 5 and 6. PCR amplifications were carried out on PTC200 (BIO-RAD laboratories, Hercules, Calif.) thermocycler. PCR products were run on 1% agarose gels for control. DNA sequencing was performed (Sequencing Core Facility INSERM/IFR26, Nantes, France) using a 48-capillary AB 3730 automatic system (Applied Biosystems, Foster City, Calif.) and analyzed using ChromasPro 1.5 software (Digital River GmbH, Shannon, Ireland).

RNA Isolation, RTPCR and Quantitative Real-Time PCR

Total RNA was isolated using the Trizol reagent (Invitrogen). After phenol-chloroform extraction and ethanol precipitation, total RNA (2 μg) was treated with RNase-free Turbo-DNase (Ambion) before reverse transcription (RT). Treated RNA was then reverse transcripted with mMLV reverse Transcriptase (Invitrogen) according to the manufacturer's instructions. RTPCR for the detection of transcripts for MICA WT and isoforms were run for 35-40 cycles (Tm: 56° C.) using the following primers: MICA5UTR/5′->3′=GTC GGG GCC ATG GGG CT (SEQ ID NO:37), MICA3UTR/5′->3′=TCA TAG GTC AGG AAA CTG AGG (SEQ ID NO:38).

Real-time quantitative PCR was performed in an ABI PRISM 7900 sequence detection application program using labeled TaqMan probes (Applied Biosystems). The following commercial ready-to-use primers/probe mixes were used (Applied Biosystems): MICA (HS00792_m1), hypoxanthine guanine phosphoribosyl transferase (HPRT, H99999909_m1) was used as an endogenous control to normalize RNA amount. Relative expression between a given sample and a reference sample was calculated according to the 2-Ct method, where the reference represents one-fold expression, as previously described (Livak and Schmittgen, 2001).

Immunoblotting

Cells were lysed on ice in 20 mmol/L Tris-HCl (pH 7.4), 137 mmol/L NaCl, 0.05% Triton X-100, 1 mmol/L supplemented with protease inhibitors (PIC, Sigma-Aldrich). Deglycosylation with Endoglycosidase H and Peptide:N-glycosidase F (Sigma-Aldrich) was performed as we described previously (Coupel et al., 2007). Cell lysates (20 μg) or culture supernatants (15 μl) were resolved by SDS-PAGE (12%) and subjected to immunoblot analysis using specific antibodies for ant-FLAG M2, MICA/MICB (BAMO1), or GAPDH as primary antibodies and secondary horseradish peroxidase-labeled anti-mouse antibodies (Cell Signaling Technology, St Quentin-en-Yveline, France). Antibody-bound proteins were detected using an ECL kit (Amersham) and luminescent image analyzer LAS-4000 (Fujifilm, Tokyo, Japan). Image analysis was performed with Multi Gauge software (Fujifilm).

Flow Cytometry

For phenotype analysis, cells (1-2 10⁵ cells/sample) were harvested, washed twice with PBS containing 1% BSA and 0.1% NaN3, and then incubated on ice for 30 min with a saturating concentration of first antibody. For intracellular staining (Flag, IFNg), cells were previously fixed in 1% paraformaldehyde, permabilzed with saponin (0.5%) before incubation with antibodies in the presence of saponin (0.1%). After three washes, cells were incubated with a PE- or FITC-labeled F(ab′)2 IgG (Jackson Lab.) at 4 C for 30 min. Cells were fixed in 1% paraformaldehyde. Negative controls were performed using an istotype-matched IgG control.

Fluorescence was measured on 10,000 cells/sample using a fluorescence activated cell sorter (FACScantoII®: Becton Dickinson, Mountain View, Calif.) and analyzed using FlowJo® software (Tree Star, Inc. Ashland, Oreg.). Data are depicted in histograms plotting median ou geomean fluorescence intensity (MFI) on a four-decade logarithmic scale (x-axis) versus cell number (y-axis).

Immunofluorescence and Confocal Microscopy

ECs were grown to confluence on glass coverslips. Cultures were washed with PBS and fixed for 20 min in 4% paraformaldehyde. Cells were incubated ON at 4° C. with blocking buffer (4% BSA in PBS) and then incubated with an anti-MICA (AMO1) mAbs or NKG2D-Fc (both 10 μg/mL) for 1 h. Cells were then incubated with FITC-conjugated goat anti-mouse or anti-human antibodies (5 μg/mL, Jackson Lab.) for 1 h. Nuclear staining was performed using Draq5 (Biostatut Ltd, Shepshed, UK). Slides were washed in PBS and mounted with ProLong® antifade reagent (Molecular Probes). Fluorescence microscopy was performed with a Nikon DM-IRBE laser scanning confocal microscope (Nikon instruments Inc., New York, USA), using a 63×1.4 oil p-aplo lens and analyzed using NIS Element Viewer™ software.

Results

1. Identification of 5 Novel Splice Transcripts and Isoforms for MICA in Human Endothelial Cells.

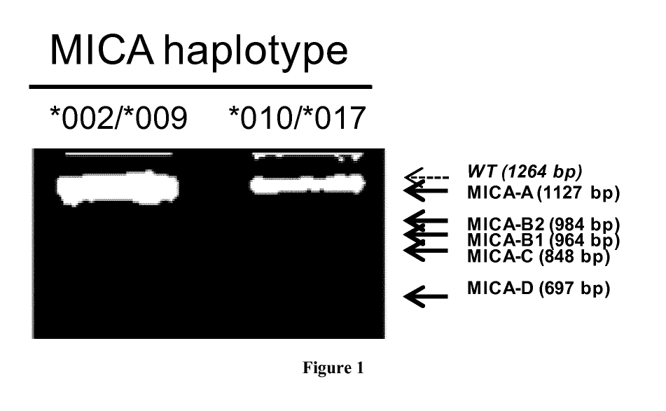

By investigating MICA transcript in cultured endothelial cells using RT-PCR we observed the presence of additive PCR products amplified using a primer pair encompassing the full length coding sequence for MICA (1216 bp for primer sequences, see “material and methods”). Five additive transcripts were found ranging from 688 bp to 1118 bp. A representative set of the 5 alternative transcripts obtained by RT-PCR, separated by electrophoresis and stained by ethidium bromide is shown in the FIG. 1. PCR products were extracted from agarose gel, purified and nucleotide sequences were determined after subcloning into Strataclone® PCR cloning vector.

The human MICA gene structure includes six exons. The original translation initiation methionine (ATG) of the MICA is located in exon 1 at position 1. Structurally, exon1 encodes a leader peptide while exon2, exon3 and exon4 code for the 3 extracellular domains α1, α2, α3 respectively. The transmembrane (TM) domain is encoded in exon5 while exon 6 encodes the intracytoplasmic (CYT) domain and an3′ untranslated region (UTR).

Analysis of the sequences indicated the following:

Isoform MICA-A:

DNA sequence includes 1118 bases. Exons1 and 2 are conserved but exon 3 is partially deleted between position 325 and 463. Deletion generates a change in ORF leading to a premature stop codon at position 637. Sequence predicts a protein of 189 AA with conserved leader peptide and α1 domain followed by a new polypeptide region (104AA) with no sequence homology in the databases.

Isoform MICA-B1:

DNA sequence includes 955 bases. Similar to MICA-A, exons 1 and 2 are conserved but exon3 and exon4 are partially deleted between the positions 325(AA86) and 463(AA132). However, a second deletion (between AA positions 220 and 275) rescue the ORF and allow normal DNA sequences for exon5 and exon6. Predicted protein for MICA-B1 isoform contains 267AA and comprises conserved leader peptide, α1 domain, TM and CYT domains but includes a novel polypeptide region instead of domains α2 and α3. This novel domain includes 91 AA and exhibits no sequence homology in the databases.

Isoform MICA-B2:

DNA sequence includes 975bases and is homologous to the wild type MICA sequence with the exception that exon 4 is deleted probably resulting from exon4 skipping during splicing. Consequently, this alternative transcript corresponds to a MICA isoform of 272AA (30.4KDa) similar to the wild type protein but with no α3 extracellular domain.

Isoform MICA-C:

DNA sequence includes 839 bases. Similar to MICA-A, MICA conserved sequences only include exon1 and exon2 encoding leader peptide and α1 domain. Partial deletion within exon3 between position 325 (AA86) and 463 (AA132) causes a change in ORF generating a new sequence and a premature stop codon in exon5. Exon 4 is completely deleted. Interestingly, due to change in ORF, ALA repeats (n=9) are replaced by CYS repeats (n=9) in the polymorphic region of exon5. Sequence predicts a protein of 174AA (19.7 kDa) with conserved leader peptide and α1 domain and a novel polypeptide region but no α2 and α3 domains, TM and CYT regions. The novel protein sequence includes 89AA and exhibits no sequence homology in databases.

Isoform MICA-D:

DNA sequence includes 688 bases. This alternative transcript displays a complete deletion of exon3 and exon4, most probably resulting from exon skipping, with a conserved ORF. Consequently, the predicted MICA-D isoform possess 176AA (19.0 kDa) and comprises conserved leader peptide, α1 domain, TM and CYT regions but no α2 and α3 domains.

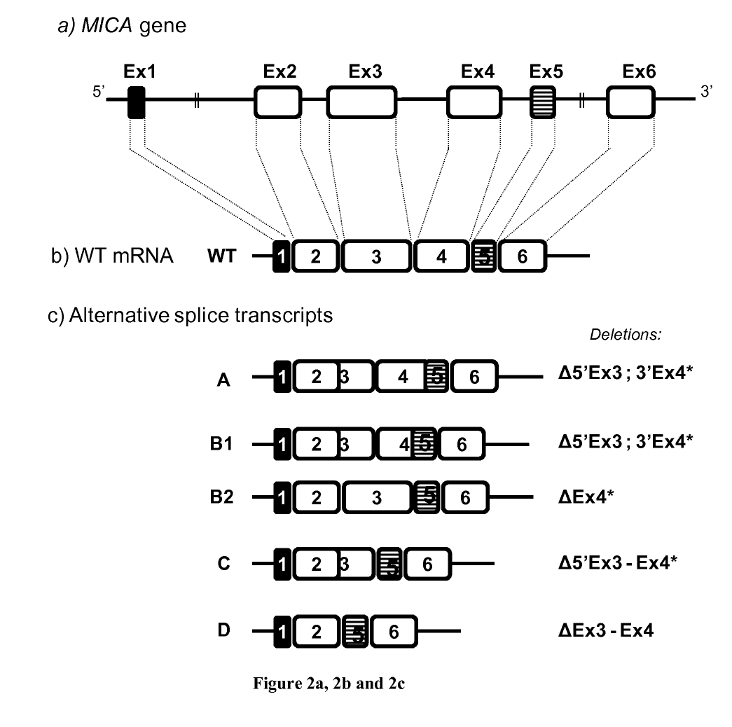

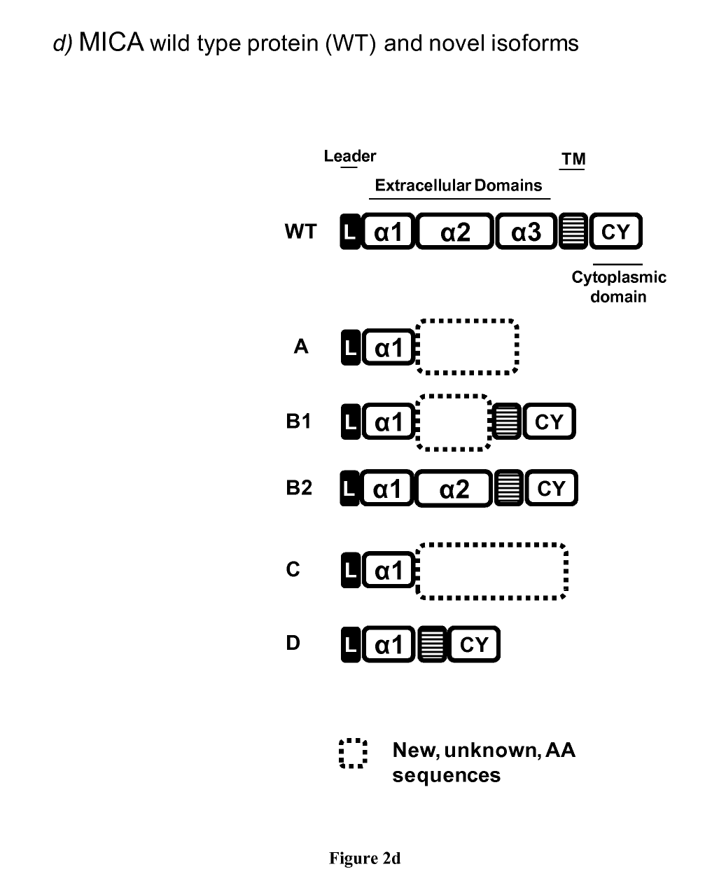

Comparison of novel AA sequences generated in isoforms MICA-A, B1 and C showed that these isoforms share partial sequence homology. A schematic representation of the alternative transcripts and predicted proteins is shown in the FIG. 2 and a summary of mRNA and protein features is reported in the Tables 1 and 2.

TABLE 1
Basic features of MICA splicing variants and their
predicted protein products

| | | | Predicted | |
|---|---|---|---|---|
| | Full cDNA | | mature | |
| | Exon1 | Transcripts | protein length | Predicted |
| | to | (ATG-stop | (w/o leader | Molecular |
| | Exon6 | codon) | peptide) | weigth |
| MICA-A | 1188 bp | 945 bp | 189 AA | 21.2 kDa |
| MICA-B1 | 1040 bp | 797 bp | 265 AA | 29.4 kDa |
| MICA-B2 | 1062 bp | 819 bp | 272 AA | 30.4 kDa |
| MICA-C | 925 bp | 682 bp | 175 AA | 19.7 kDa |
| MICA-D | 774 bp | 531 bp | 176 AA | 19.0 kDa |

TABLE 2
Biochemical characteristics of MICA WT and isoforms

| | N- | N- | PKC | CK2 | |
|---|---|---|---|---|---|
| | glycosylation | myristoylation | phosphorylation | phospho | |
| MICA | sites | sites | sites | sites | others |
| MICA WT | 8 | 6 | 6 | 3 | Disulfide |
| | (31-34) | (39-44) | (27-29) | (112-115) | (225-282) |
| | (79-82) | (243-248) | (126-128) | (212-215) | Camp |
| | (125-128) | (262-267) | (155-157) | (336-339) | phospho |
| | (210-213) | (288-293) | (224-226) | | sites |
| | (220-223) | (375-380) | (238-240) | | (332-335) |
| | (234-237) | (378-383) | (297-299) | | (333-336) |
| | (261-264) | | | | |
| | (289-292) | | | | |
| MICA-A | 2 | 1 | 1 | 1 | — |
| | (31-34) | (39-44) | (27-29) | (154-157) | |
| | (79-82) | | | | |
| MICA-B1 | 2 | 3 | 1 | 2 | Tyr |
| | (31-34) | (39-44) | (27-29) | (154-157) | Phospho |
| | (79-82) | (280-285) | | (241-244) | site |
| | | (283-288) | | | (169-176) |
| | | | | | CAMP |
| | | | | | phospho |
| | | | | | sites |
| | | | | | (237-240) |
| | | | | | (238-241) |
| MICA-B2 | 3 | 3 | 4 | 2 | CAMP |
| | (31-34) | (39-44) | (27-29) | (112-115) | phospho |
| | (79-82) | (287-292) | (126-128) | (248-251) | sites |
| | (125-128) | (290-295) | (155-157) | | (244-247) |
| | | | (204-206) | | (245-248) |
| MICA-C | 2 | 2 | 1 | 3 | CYS rich |
| | (31-34) | (39-44) | (27-29) | (154-157) | domain |
| | (79-82) | (213-218) | | (204-207) | (223-238) |
| | | | | (217-220) | |
| MICA-D | 2 | 3 | 1 | 1 | CAMP |
| | (31-34) | (39-44) | (27-29) | (152-158) | phospho |
| | (79-82) | (191-196) | | | sites |
| | | (194-199) | | | (148-151) |
| | | | | | (149-152) |

2. Alternative Splice Transcripts Associate with Alleles MICA *015 and *017 and Result from a Point Deletion in Intron4 Splice Donor Site.

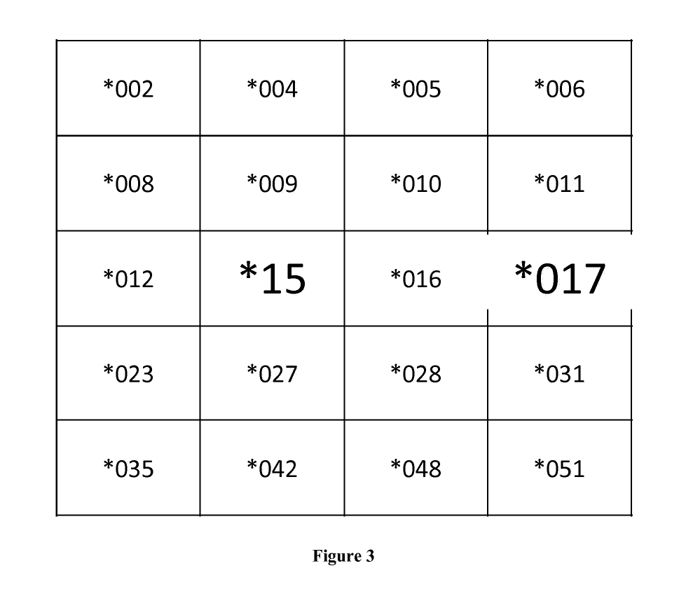

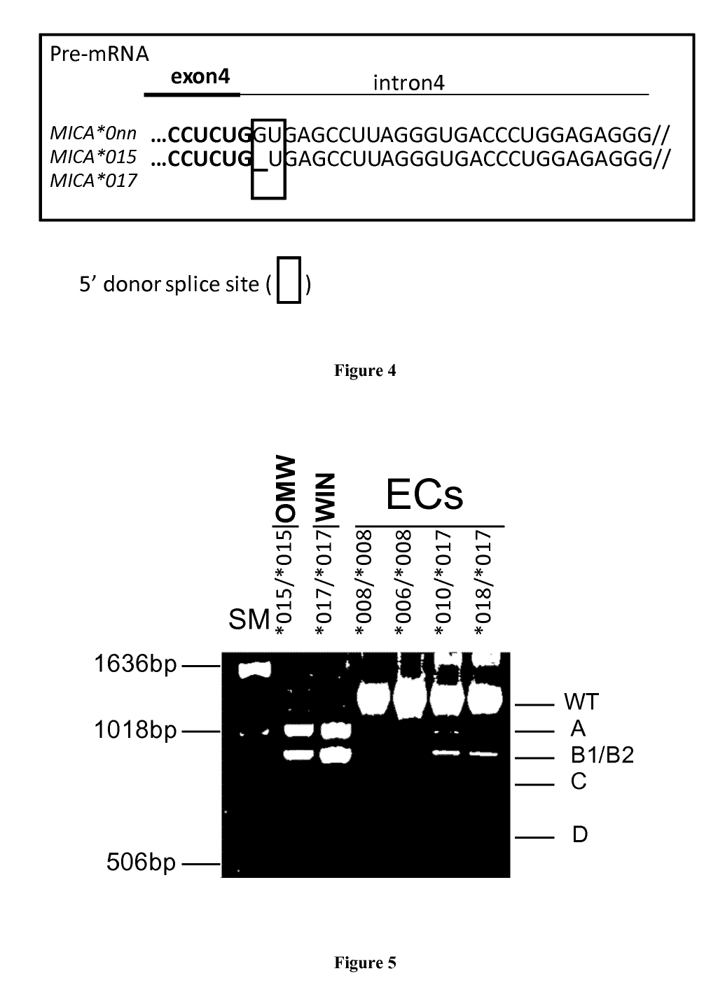

To investigate whether MICA gene polymorphism accounts for the occurrence of the novel MICA transcripts, haplotypes of MICA in our cultures of human endothelial cells were determined by PCR and sequencing as we previously reported (Tonnerre P. et al., Transpl. Proc. 2010, Tonnerre P. et al JASN 2013). Alternative MICA transcripts were found associated with two MICA alleles *015 and *017 out of the 22 alleles tested (see FIG. 3). No alternative transcripts were found associated with the most frequent MICA alleles (MICA*002, *004, *008, *009, *011 . . . ). The presence of a repeat of 9 alanines (A9) except for MICA-C (where alanine repeats are replaced by cysteine repeats (n=9) also argue in favor with an association of the AST with the alleles *015 and *017 that both contain the A9 repeat. Sequencing of DNA obtained by PCR amplification of MICA introns and exons from cells carrying the *015 or *017 MICA alleles revealed a point deletion of the first 5′base (G) of the intron4 donor splice site (FIG. 4). Consistent with these findings previous studies have reported on MICA gene mutation or deletion at the exon4/intron4 junction (Obuchi et al., 2001). Nevertheless, to our knowledge, we provide here the first evidence for a link between this mutation and the presence of alternative splicing transcripts associated with both alleles MICA*015 and *017. Indeed, deletion in the intron4 donor splice site is consistent with alternative splicing. Alternative splicing can alter the mRNA product in several ways. At the simplest level, an exon can be removed (exon skip), lengthened or shortened (alternative 5′AS or 3′AS splicing). Thus, observed mechanisms of alternative splicing alteration include exon skipping, intron retention, and the use of an alternative splice donor or acceptor site. Here, we speculate that partial deletion of the donor splice site induces exon 3 and/or 4 skipping (MICA-B2 and MICA-D) as well as alternative 5′AS or 3′AS (MICA-A, B1, C). To confirm the association of AST with the genetic variant*015 and *017, the presence of the AST was assessed by RTPCR on mRNAs issued from ECs (n=2) heterozygous for both alleles. As illustrated in the FIG. 5, alternative transcripts were detected in all samples. We also found, AST in two lymphoblastoid cell lines OMW and WIN homozygous for MICA*015 and *017, respectively. Thus, the presence of AT in ECs from MICA*015 or*017 individuals but also in lymphocytes suggests that these MICA isoforms are not restricted to a cell type (i.e. EC) but instead are expressed in both hematopoietic and non hematopoietic cells. Importantly, in cells homozygous for the mutation and expressing MICA AT no mRNA for MICA WT was detected.

Next, we sought to determine the allelic frequency for MICA*015 and *017 in a population of healthy Caucasian donors (n=186). MICA genotyping identified 12 MICA*017 alleles out of 372 tested (allelic frequency: 3.22%), corresponding to 12 donors carrying one MICA*017 allele. A single, heterozygous, carrier of MICA*015 allele was found in this population (allelic frequency: 0.27%). Consistent with previous reports (Xu et al., 2012), these data establish MICA*015 and *017 as rare and low frequency alleles, respectively.

3. Establishment of a Dedicated Method for the Detection and the Quantification of the Five MICA Alternative Transcripts in Samples by RT-PCR.

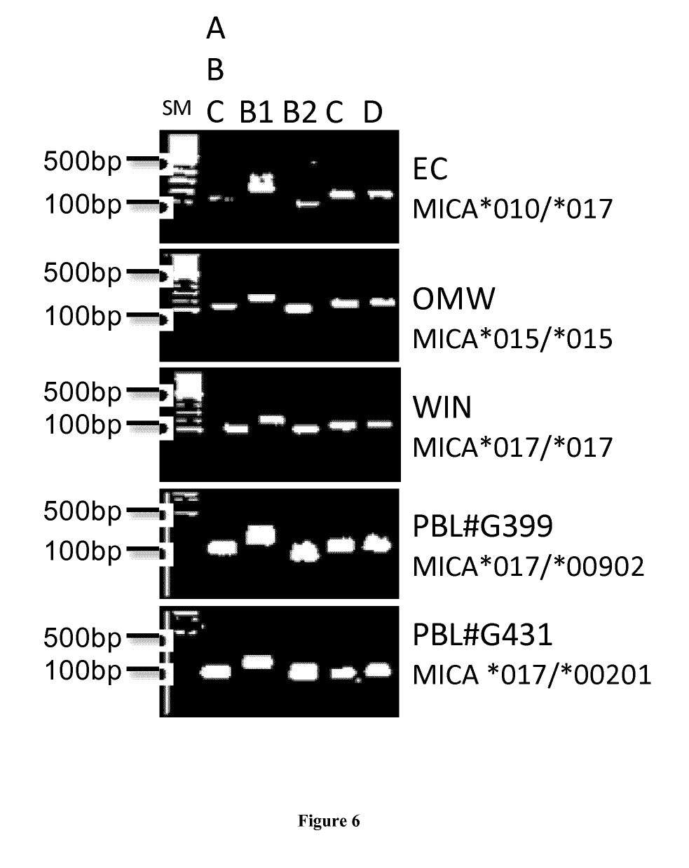

In order to allow the rapid detection and the quantification of the five alternative transcripts MICA-A, -B1, -B2, -C, -D in biological samples we sought to develop a PCR assay with dedicated primers for the selective amplification of the alternate transcripts in cell or another biological samples. We were able to design specific primers to discriminate all MICA-isoforms from wild type MICA with the exception of MICA-A. Consequently, a generic primer for MICA-A, -B1, -B2, -C was used. Sequences of the primer pairs designed for quantitative real time PCR (QPCR), length of PCR product and specific Tm are presented in SEQ ID N0:16-25. To validate the RT-PCR assay, RT-PCR were run on a panel of cells containing at least one allele MICA *015 or *017. These cells include one endothelial cell culture (EC), the cell lines WIN and OMW and PBLs from 2 donors. Representative PCR experiments are illustrated in the FIG. 6 and they established the reliability of our PCR assay to detect and to quantify (data not shown) the alternative transcripts in cells.

4. Cloning and Expression of MICA Isoforms in Transfected Cells.

It was initially established that the expressed MICA, encodes membrane-bound polypeptides of 365 amino acids, with a relative molecular mass of 43.1 kDa. The transmembrane domain of the MICA molecule is encoded by exon 5 that displays a microsatellite polymorphism defining at least six specific variants which differ in the number of polyalanine repeats (GCT) inserted at AA position 296. To characterize the novel MICA isoforms, full cDNAs for each alternate transcript were produced and cloned into a pCMV-TAG 4A FlagM2 plasmid for transfection experiments. COS-7 cells were transfected and recombinant isoforms were analyzed by Western blots, flow cytometry and cellular immunostaining Firstly, expression of the transfected alternate transcripts was detected by RT-PCR using a dedicated set of isoform-specific primers. Unlike for other AST, we were unable to design specific primers to discriminate MICA-A from other AST. Consequently, a generic primer for MICA-A, -B1, -B2, -C was used. Sequences of the primer pairs designed for quantitative real time PCR (QPCR), length of PCR product and specific Tm are presented in the SEQ ID N0:16-25

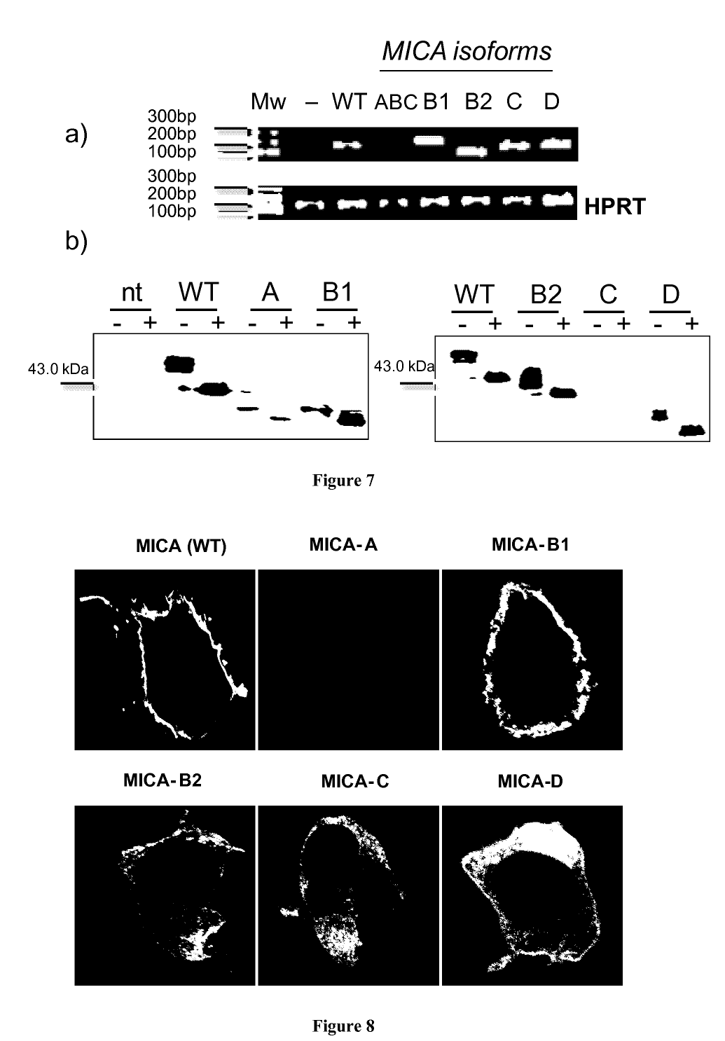

As a result of PCR analyses, we were able to detect significant levels of mRNA for all thefive alternative transcripts (FIG. 7a).

In parallel experiments, transfected cells were lyzed for biochemical studies. Cell lysates were resolved by SDS-PAGE and immunoblotted with an anti-Flag antibody. A representative Western blotting is provided in the FIG. 7b and reveals that all isoforms with the exception of MICA-C are detected. Unlike other isoforms, MICA-A seems to be expressed at a low rate that could suggest a lack of protein stability. Deglycosylation with PNGase F was performed. Relative molecular weights of the isoforms are in the range of estimated molecular weights calculated from the predicted AA sequences.

Next immunostaining with an anti-Flag antibody and flow cytometry analysis confirmed that most isoforms are express as stable proteins in transfected cells with the exception of MICA-A that was not significantly detected by this method. In contrast, cells expressing MICA-C were consistently found. These data could confirm that MICA-A alternate transcripts do not lead to a stable protein. In contrast, although not detectable by Western blot, MICA-C seems to gives rise to a stable protein in transfectants.

These results were further confirmed by immunohistochemistry on transfected cells. Indeed, cell imaging analysis of the transfectants clearly demonstrated the expression of all isoforms except MICA-A. Next, protein localization was examined after immunostaining by confocal microscopy. Consistent with the predicted sequences, the lack of TM and CYT domains in MICA-A and MICA-C impairs their expression at the cell surface. As expected, MICA-B1, -B2 and -D that possess intact TM and CYT domains, were found located at cell membrane. MICA-B2 and -D were also additionally found in the cytoplasm of transfected cells. (FIG. 8).

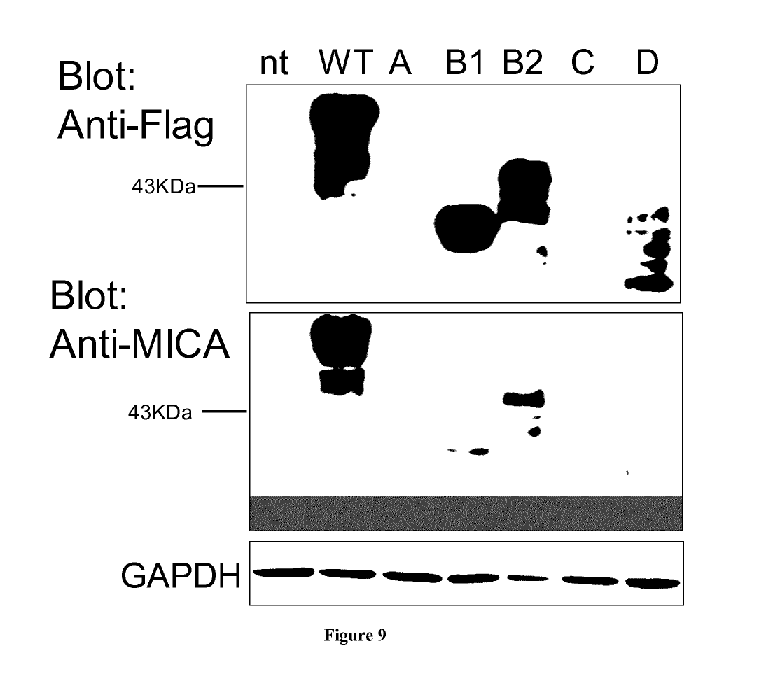

Wild type MICA proteins can be released from the cell membrane as a result of proteolysis mostly achieved by the adamalysin ADAM10 ((Groh et al., 2002) (Salih et al., 2002) (Salih et al., 2008) (Waldhauer et al., 2008) although other metalloproteinases could also be involved (Sun et al., 2011). Consequently, cell expressing high level of MICA can produce a soluble form of MICA resulting from the enzymatic cleavage of the membrane bound proteins. This process impairs activation of effector cells expressing NKG2D and thus is largely involved in some pathological conditions including tumor and metastasis progression ((Holdenrieder et al., 2006)). Cleavage site for ADAM10 is located within the α3 extracellular domain of MICA (Wang et al., 2009). Deletion of the α3 extracellular domain of MICA in all five isoforms strongly suggests that shedding of the isoforms could not operate. Moreover, palmitoylation of two cystein residues (Cys306 and Cys307) in the intracellular domain of MICA is necessary for the recruitment of MICA in the membrane cholesterol-enriched microdomains that promotes proteolytic shedding of MICA (Aguera-Gonzalez et al., 2011). These two cysteins are conserved in isoforms MICA-B1, -B2, -D but are absent in isoforms MICA-A and C. To test whether the novel isoforms can be released as soluble forms, transfectants were treated with a selective inhibitor of ADAM10 (GI254023X, kindly provided by GSK) before facs analysis. No significant increase in isoform expression at the cell surface was observed, as expected, suggesting that no ADAM10-dependent proteolytic cleavage of the isoforms occurred. In contrast, cells transfected with the wild type, full length MICA cDNA expressed higher level of MICA (30% of increase) in the presence of ADAM10 inhibitor. We cannot exclude the possibility that other metalloproteinases could operate. To test this hypothesis, western blots were performed on cell culture supernantants to determine whether MICA isoforms can be released from transfectants into the extracellular medium. FIG. 9 illustrates our findings that suggesting that at least one isoform, MICA-B1, is released as a soluble form in the culture supernatants.

Together, the above experiments indicate that 4 out the 5 AST that we identified give rise to stable proteins in transfectants, 3 (MICA-B1, -B2, -D) are anchored in the cell membrane, and 2 (MICA-A, -C) seem to be retained in the cytoplasm.

5. Biological Activity and Immune Properties of MICA Isoforms.

a. Isoforms Detection Using Specific Anti-MICA Antibodies/Detection of MICA Isoforms Using Anti-MICA Antibodies.

To determine whether deletions observed in the new MICA proteins enable their detection by conventional analysis anti-MICA/B (AMO1) mAb was incubated with the transfectants. Analysis of antibody reactivity was determined by flow cytometry. Our results indicate that anti-MICA antibodies directed against the α1 and α2 domains are able to bind only to MICA-B2 expressed on transfectants. This result was further confirmed by fluorescence microscopy and confocal analysis. These data are consistent with the lack of α2 domain in other isoforms. These data also suggest that isoform B2 is structurally close enough to wild type MICA proteins to be detected with usual detection assay while other cell-bound isoforms (namely B1 and D) could be detected only by antibodies specific for the α1 domain or for the new polypeptide sequence (MICA-A, -B1, -C). Moreover, immunogenicity of the isoforms seems to differ according to the isoforms (see§5b).

b. Recognition of MICA Isoforms by Human Anti-MICA Antibodies in MICA Sensitized Transplant Recipients

Consistent with the high polymorphism of MIC molecules, specific antibodies against MICA have been reported in the serum of patients who had rejected kidney allografts, suggesting a potential role for these molecules in transplant immunopathology (Sumitran-Holgersson et al., 2002; Zwirner et al., 2000) (Amezaga et al., 2006) (Terasaki et al., 2007). Renal and pancreatic grafts with evidence of both acute and chronic rejection have been shown to express MIC proteins, and anti-MIC antibodies have been identified in the serum of these patients. Expression of MICA and MICB in transplanted organs has been demonstrated (Hankey et al., 2002).

Consequently, we investigated the potential clinical relevance of the isoforms in the context of MICA sensitization in kidney transplant recipients. To this aim sera from transplanted recipients containing anti-MICA antibodies were incubated with the COS cells transfected with the different isoforms. Mock transfected cells and control sera (a pool of AB sera from male donors) were used as negative controls. MICA reactivities and specificities of the sera were previously determined. As a result we found that anti-MICA antibodies in the sera of sensitized transplant recipients are able to bind to MICA-B2 suggesting that this isoform could play a role in an allogeneic response in organ transplantation.

c. MICA-B2 is a New Ligand for NKG2D

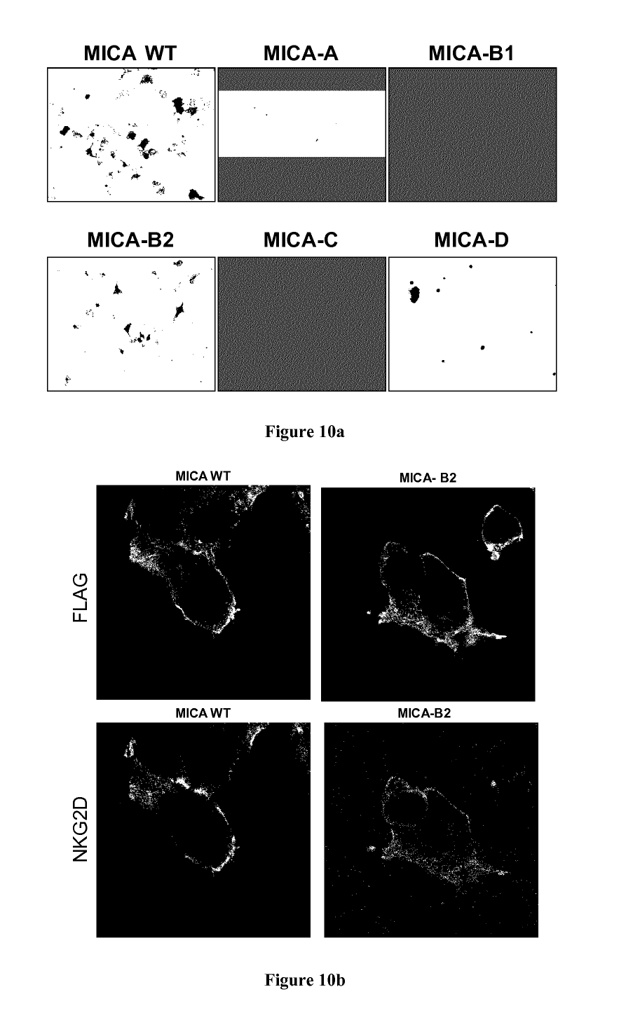

To functionally assess a role for the MICA isoforms in immune regulation, the possible interaction of MICA isoforms with NKG2D receptor, the natural receptor of full length MICA, was investigated by facs and by cellular immunofluorescence. Together, facs analysis and immunocytology reveals that MICA-B2 binds to a recombinant NKG2D-Fc protein (FIG. 10a). Staining was located at the cell membrane in accordance with the localization of MICA-B2 established above as established by colocalisation of both NKG2D and Flag staining observed by confocal microscopy (FIG. 10b). Although no significant staining was found for other isoforms expressed at cell surface (li.e. MICA-Bland MICA-D), we cannot exclude the possibility that these isoforms could interact with NKG2D receptor through their aldomain. If such binding occurs, its affinity could be not high enough to allow detection in our experimental system.

### DISCUSSION

NKG2D displays only limited sequence similarity to other NKG2 family members and CD94 (20-30% of homology), has not been demonstrated to directly interact with MHC class I proteins and forms homodimers (Li et al., 2001; Steinle et al., 2001). NKG2D receptor is expressed on all NK cells, CD8 αβTCR T cells, and γδTCR T cells (Bauer et al., 1999), implying a broad role in immune responses. NKG2D can deliver an activation signal to NK cells and, under some conditions, can act as a costimulatory receptor for TCR-mediated activation of T cells, in a similar manner as CD28 (Groh et al., 2001) (Ehrlich et al., 2005).

NKG2D ligands include MICA, MICB and ULBPs (1-6) in humans (Bahram et al., 1996) (Kubin et al., 2001). These ligands share some structure and sequence homologies with classical HLA class I (Bahram et al., 1996; Groh et al., 1996). Structurally, MICA encompass 3 immunoglobulin-like alpha domains: α1, α2, α3 domains similar to alpha domains in HLA-A, -B and -C. In contrast to classical HLA class I, MICA as well as other NKG2DL don't bind β2microglobulin (β2-m). MIC proteins do not require either peptide or β2-m for stability or cell-surface expression (Groh et al., 1996).

Here we described five novel isoforms for the non classical MHC molecule MICA. We demonstrate that these MICA isoforms result from alternative splicing transcripts caused most probably by a deletion of the first base (G) of intron 4 splice donor site. This deletion induces exon3 and exon4 skipping and/or partial or total deletion. We found this deletion of intron 4 splice donor site associated with at least two alleles: MICA*015 and *017. Although we cannot exclude the possibility that other alleles carry this mutation, neither the deletion in intron 4 nor the presence of AST was detected in the most frequent MICA alleles (MICA*002, *004, *008, *009, *011 . . . ). Initial identification of the alleles MICA*015 and *017 reported on a guanine deletion at the 3′ end of exon 4 as a common feature for both alleles (Obuchi et al., 2001). It was subsequently speculated that this change will not affect the splicing pattern, because the splicing consensus sequences are still conserved, but rather it will cause a frameshift in mRNA. However, our findings mostly suggest that the deleted guanine was in fact the 5′ end guanine of intron 4 splice donor site. The presence of alternative splicing transcripts that we found associated with both alleles strongly supports this hypothesis. Importantly, in cells homozygous for MICA*015 or *017 alleles, no full length transcript for MICA was found. Although no pathology was clearly associated with MICA alleles *015 and *017, previous reports indicated that MICA A9 repeat was found proposed as a possible genetic risk factor for psoriasis (Gonzalez et al., 1999) (Romphruk et al., 2004). Interestingly, MICA*017 and MICA*015 were exclusively found in panels that carry HLA-B*57 and -B*45, respectively (Obuchi et al., 2001). Consistently, in our study both EC donors heterozygous for MICA*017 also carry one HLA-B*57 allele.

Among the five isoforms that we identified in the present study, two, MICA-A and MICA-C, lack TM and CYT domains, thus are not expressed at the cell membrane but instead seem retained in the cytoplasm. No intracellular function has been reported yet for NKG2DL. However, regulatory mechanisms of NKG2DL are multiple and include both transcriptional, translational, posttranslational processes (for review see (Champsaur and Lanier, 2010)). Regulation by miRNAs targeting the 3′UTR of MICA has been reported (Stern-Ginossar and Mandelboim, 2009). Although, the functions that MICA-A and MICA-C could play still remain to be explored, one can speculate that these MICA isoforms, which retain wild type 3′UTR, could to target miRNAs by providing additional mRNA target. The possible regulatory functions of these new isoforms need investigation. The function of the new polypeptide encoded in C/N-term of the α1 domain as well as the possibility that these proteins can be released or secreted in the extracellular medium have to be tested.

MICA-B1, B2 and MICA-D are expressed at the cell membrane and could potentially interact with the NKG2D receptor. The stochiometry of the NKG2D/NKG2DL complexes is 2:1: one NKG2D homodimer binds a single monomeric NKG2DL, either H60, RAE-1 or MICA (Strong, 2002). Each NKG2D monomer (NKG2D-A and -B) predominately contacts either the α1 or α2 domains of MICA or ULBP, with the two sub-site interactions contributing approximately equally to the overall interaction, unlike the CD8/MHC class I interaction (Strong, 2002).

Here we provide the first evidence that isoform MICA-B2 is a new ligand for NKG2D. Interestingly, the overall structure of MICA-B2 resembles the ULBP rather than the MICA in that it contains only α1 and α2 domains. While ULBP 1, 2, 3 are GPI linked proteins, other ULBP (4 and 5) are transmembrane proteins. Thus, structurally, MICA-B2 isoform featured by only α1 and α2 domains, transmembrane and cytoplamic domains is closed to ULBP4 and 5. The functional ability of ULBPs to trigger a NKG2D-dependent activating signal in NK cells established the basis for NKG2D engagement in the absence of a3 domain and supports the idea that MICA-B2 is a functional ligand for NKG2D. We already showed that MICA-B2 efficiently binds NKG2D in vitro. Thus we can hypothesize that MICA-B2 can play a role in immune processes. Importantly, the lack of α3 domain strongly impairs shedding by metalloproteinase ADAM10/erp5 suggesting that expression of this MICA isoform could more stable that the WT MICA. Owing the importance of soluble MICA in immune evasion processes in cancer and infections, this feature could be of importance. Moreover, our findings also establish that polyreactive sera from MICA sensitized transplant recipients are able to bind to MICA-B2 expressed on COS transfected cells. In support with a role for MICA-B2 in transplant immunology, peptides previously reported to be immunogenic and located in the α2 domain (Suarez-Alvarez et al., 2009a) are conserved in this isoform. Binding was similar to the one observed for MICA*02 transfectants.

Substitutions distant from the interaction surfaces may also affect binding, presumably through indirect conformational changes. The methionine-to-valine substitution at position 129 in the α2 domain of MICA, a conservative substitution which has no atom closer than 21 Å to any atom of NKG2D, has been experimentally shown to have a 30-fold affect on the affinity for NKG2D (Steinle et al., 2001). In our samples, MICA*017 allele associates with a methionine at position 129 suggesting a possible high affinity of the MICA-B2 isoform for NKG2D receptor. This position was not conserved in the other four isoforms. Also, some of the characterized MIC allelic differences are known to dramatically affect folding and cell-surface expression of MICA. The arginine-to-proline substitution at position six in MICA010 illustrates the impact of proline substitution that disrupt the platform β-sheet, with as a result no protein expression for this allele (Li et al., 2000). It remains unclear exactly how NKG2D can tolerate such plasticity in ligand binding sites while retaining specificity and significant affinities.

Yet, no interaction with a recombinant NKG2D-Fc chimera protein was observed in our experimental conditions for MICA-B1 and MICA-D. We can rule out the possibility that an interaction occurred but was not detected in our experimental system.

Our preliminary data from immunoblotting suggest that MICA-B1 can be produced or released as a soluble form in culture supernatant. Release of soluble MICA has been shown to be a major process triggering immune escape in cancer (Groh et al., 2002) and a valuable prognostic maker of tumor outcome (Holdenrieder et al., 2006). Soluble isoforms have been previously reported for HLA-G (Ishitani and Geraghty, 1992; Paul et al., 2000). and ULPB (Cosman et al., 2001). Four novel functional splice variants of ULBPs including ULBP4-I, ULBP4-II, ULBP4-III and RAET1G3 have been reported recently (Cao et al., 2008). All ULBP4 splice variants (ULBP4-I, ULBP4-II and ULBP4-III) were type 1 membrane-spanning molecules and had the ability to bind with human NKG2D receptor in vitro. In contrast to soluble MICA that downregulates NLKG2D expression through internalization, soluble forms of ULBPs bind to NKG2D and activate intracellular signaling via protein tyrosine phosphorylation, and activation of the Janus kinase 2, STATS, mitogen-activated protein kinase, and phosphatidylinositol 3-kinase (PI 3-kinase)/Akt pathways (Sutherland et al., 2002). The biological activity of soluble MICA-B1 remains to be established.

The functional impact of isoforms containing only the α1 domain (MICA-D) remains to be established. We would like to test the hypothesis that these isoforms could be NKG2D antagonists by interacting with the NKG2D receptor without engaging signaling. Blockade of NKG2D has been proposed as a therapeutic approach to avoid autommune disease (Ogasawara et al., 2004).

Concerning the clinical relevance of our findings, although no immune disorder has been reported with HLAB57, it is important to notice that the genetic polymorphism that has the greatest impact on immune control of human immunodeficiency virus (HIV) infection is expression of HLA-B*57 (Kloverpris et al., 2012b). The mechanism for this protective effect still remains partly understood (Feinberg and Ahmed, 2012) (Kloverpris et al., 2012a). Viral control is linked to the expression of certain alleles encoding HLA class I molecules, particularly HLA-B*57, HLA-B*27 and HLA-B*5801, which suggests an immunological basis related to the function of CD8⁺ T cells. The mechanistic basis for the association remains unclear. Here we speculate that MICA polymorphism MICA*015 and MICA*017 linked to HLA-B57 could be a yet inexplored immunodominant factor involved the immune control of HIV infection.

### EXAMPLE 2

To functionally assess a role for the MICA isoforms in immune regulation, the possible interaction of MICA isoforms (B1, B2 and D) with NKG2D receptor, the natural receptor of full length MICA, was investigated further on transfected cells. Transient and stable transfectants were established in CHO and HEK cell lines, respectively. Recombinant proteins were also produced by cloning the extracellular domains of MICA isoform B1, B2 and D into a pET1histag plasmid and purified. Functional assays include NKG2D modulation, NK cell activation (CD107a and IFN gamma), intracellular calcium flux in NK cells.

MICA_B2: MICA-B2 Isoform is a NKG2D Receptor Agonist Ligand:

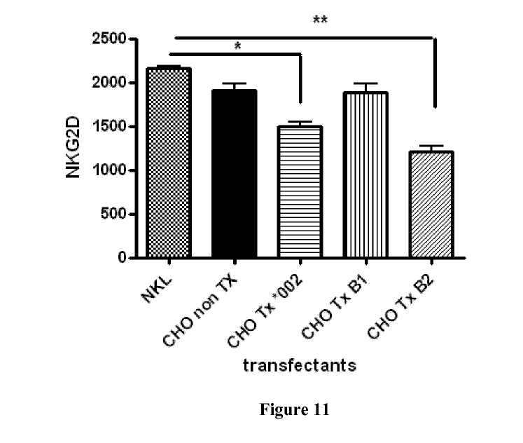

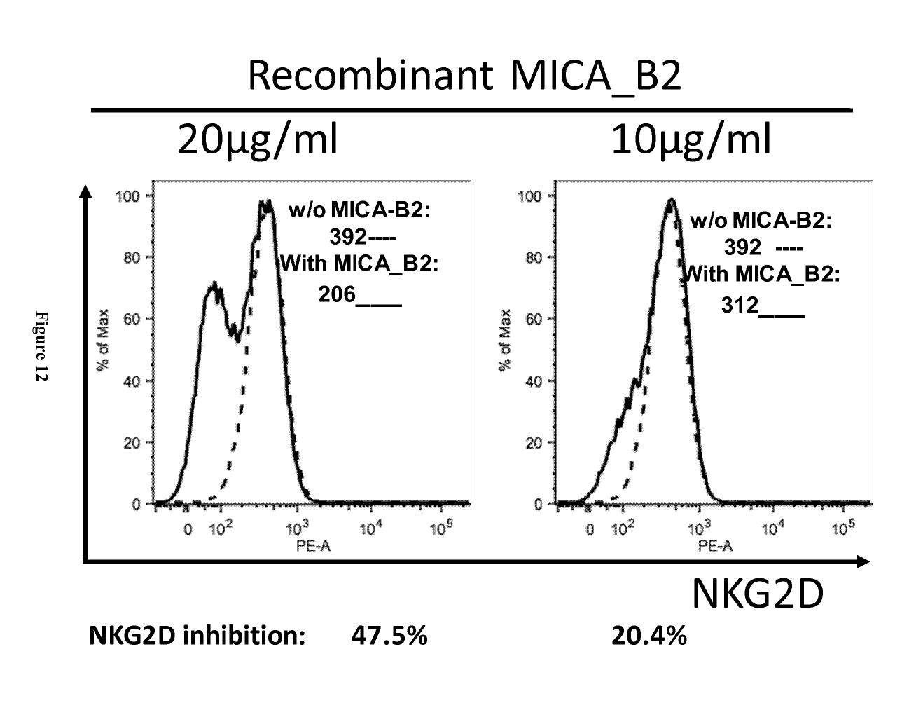

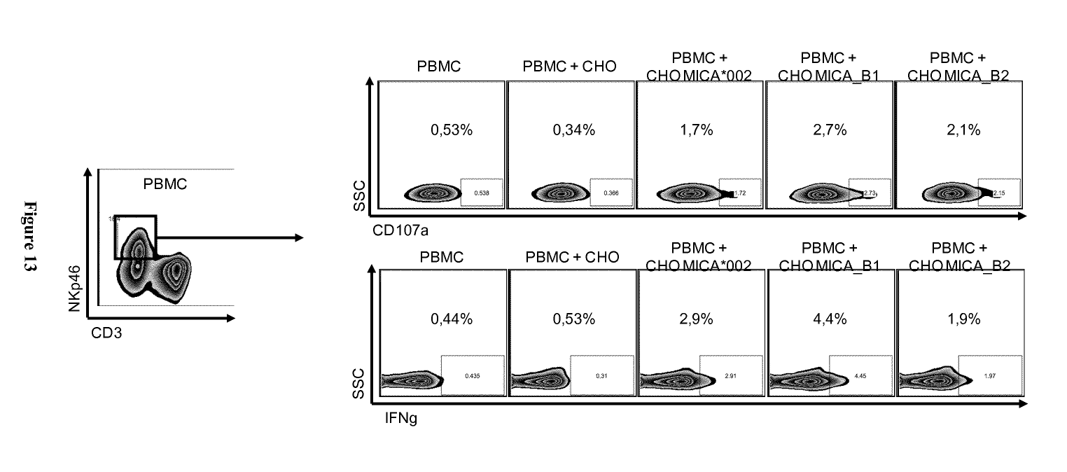

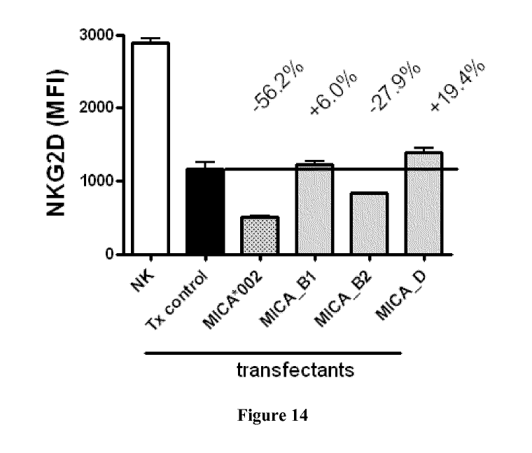

As show in FIG. 11, Transfected cells expressing MICA_B2 efficiently downregulate NKG2D expression on NK cells after an overnight coculture. Downregulation induced by MICA_B2 was similar than the one obtained using transfected CHO cells expression wild type (WT) full length MICA (allele *002) used as positive control. Similar results were obtained using a purified recombinant MICA-B2 protein. In our conditions, incubation of NK cells in the presence of recombinant soluble MICA-B2 protein decreases NKG2D expression up to 47.5% compared to control (FIG. 12). Activation of NK cells was measured by the analysis of NK cell degranulation (expression of CD107a) and the production of the cytokine IFN gamma. Both analyzed by facs. As illustrated in FIG. 13, MICA-B2 transfectants activate NK cells as reflected by the induction of CD107a expression on NK and by the production of IFNg. Time lapse video microscopy further illustrate the ability of MICA-B2 to engage NKG2D receptor on NK cells and to rapidly induce an intracellular calcium flux into NK cells (data not shown). Together, the data clearly demonstrate that MICA-B2 is a new functional ligand for the activating receptor NKG2D that bind and activate NKG2D in NK cells.

MICA-B1: MICA-B1 Isoform is a Partial NKG2D Receptor Agonist Ligand:

As show in FIG. 1, CHO transfected cells expressing MICA_B1 do not significantly downregulate NKG2D expression on NK cells after an overnight coculture. In contrast, significant downregulation was induced by transfected CHO cells expressing wild type (WT) full length MICA (allele *002) used as positive control or expressing the isoform MICA_B2. Activation of NK cells was measured by the analysis of NK cell degranulation (expression of CD107a) and the production of the cytokine IFN gamma both analyzed by facs. As illustrated in FIG. 3, MICA-B1 transfectants activate NK cells as reflected by the induction of CD107a expression on NK and by the production of IFNg. Activation of NK cells by MICA-B1 was similar to those of MICA-B2. Together, the data clearly demonstrate that MICA-B1 is a new functional ligand for the activating receptor NKG2D that bind and activate NKG2D in NK cells without promoting downregulation of NKG2D receptor.

MICA-D: MICA-D Isoform is a NKG2D Receptor Antagonist Ligand:

To establish the function of isoform MICA-D, the MICA expressing epithelial cell line HEK was transiently transfected with plasmid encoding MICA-WT (*002), MICA-B1, MICA-B2 and MICA-D (FIG. 4). NK cells were incubated overnight with transfectants and analyzed for NKG2D by flow cytometry. HEK transfected with an empty plasmid, used as a negative control, efficiently decrease NKG2D expression on NK surface. HEK transfected with a plasmid coding for MICA-WT (*002) further decrease NKG2D level compared to HEK transfected with an empty plasmid indicating that NKG2D downregulation was correlated to the level of MICA expression on HEK cells and a dose-dependent regulatory effect. In comparison to MICA-WT, we found that MICA-B1 has no effect while MICA-B2, similar to MICA-WT has an additive effect on NKG2D regulation. Together these data are consistent with results obtained with stable CHO transfectants (see above) and confirm that MICA-B2 but not MICA-B1 downregulates NKG2D. HEK cells transfected with MICA-D significantly inhibit the modulation of NKG2D induced by HEK cells on NK cells. These data clearly indicate that MICA-D in contrast to MICA-B2 is an antagonist ligand for the NKG2D receptor. Our finding provide the first evidence that alpha1 domain of MICA is functionally active, bind to NKG2D and efficiently compete with full length MICA protein providing a new inhibitory/blocking ligand for NKG2D.

### REFERENCES

Throughout this application, various references describe the state of the art to which this invention pertains. The disclosures of these references are hereby incorporated by reference into the present disclosure.
Aguera-Gonzalez, S., C. C. Gross, L. Fernandez-Messina, O. Ashiru, G. Esteso, H. C. Hang, H. T. Reyburn, E. O. Long, and M. Vales-Gomez. 2011. Palmitoylation of MICA, a ligand for NKG2D, mediates its recruitment to membrane microdomains and promotes its shedding. Eur J Immunol 41:3667-3676.
Amezaga, N., M. Crespo, M. Lopez-Cobos, M. A. Millan, O. Vinas, M. Sole, F. Oppenheimer, J. Martorell, and M. G. Ercilla. 2006. Relevance of MICA antibodies in acute humoral rejection in renal transplant patients. Transpl Immunol 17:39-42.
Bahram, S., H. Inoko, T. Shiina, and M. Radosavljevic. 2005. MIC and other NKG2D ligands: from none to too many. Curr Opin Immunol 17:505-509.
Bahram, S., N. Mizuki, H. Inoko, and T. Spies. 1996. Nucleotide sequence of the human MHC class I MICA gene. Immunogenetics 44:80-81.
Bauer, S., V. Groh, J. Wu, A. Steinle, J. H. Phillips, L. L. Lanier, and T. Spies. 1999. Activation of NK cells and T cells by NKG2D, a receptor for stress-inducible MICA. Science 285:727-729.
Caillat-Zucman, S. 2006. How NKG2D ligands trigger autoimmunity? Hum Immunol 67:204-207.
Cao, W., X. Xi, Z. Wang, L. Dong, Z. Hao, L. Cui, C. Ma, and W. He. 2008. Four novel ULBP splice variants are ligands for human NKG2D. Int Immunol 20:981-991.
Champsaur, M., and L. L. Lanier. 2010. Effect of NKG2D ligand expression on host immune responses. Immunol Rev 235:267-285.
Collins, R. W., H. A. Stephens, M. A. Clare, and R. W. Vaughan. 2002. High resolution molecular phototyping of MICA and MICB alleles using sequence specific primers. Hum Immunol 63:783-794.
Cosman, D., J. Mullberg, C. L. Sutherland, W. Chin, R. Armitage, W. Fanslow, M. Kubin, and N. J. Chalupny. 2001. ULBPs, novel MHC class I-related molecules, bind to CMV glycoprotein UL16 and stimulate NK cytotoxicity through the NKG2D receptor. Immunity 14:123-133.
Coupel, S., F. Leboeuf, G. Boulday, J. P. Soulillou, and B. Charreau. 2004. RhoA activation mediates phosphatidylinositol 3-kinase-dependent proliferation of human vascular endothelial cells: an alloimmune mechanism of chronic allograft nephropathy. J Am Soc Nephrol 15:2429-2439.
Coupel, S., A. Moreau, M. Hamidou, V. Horejsi, J. P. Soulillou, and B. Charreau. 2007. Expression and release of soluble HLA-E is an immunoregulatory feature of endothelial cell activation. Blood 109:2806-2814.
Ehrlich, L. I., K. Ogasawara, J. A. Hamerman, R. Takaki, A. Zingoni, J. P. Allison, and L. L. Lanier. 2005. Engagement of NKG2D by cognate ligand or antibody alone is insufficient to mediate costimulation of human and mouse CD8+ T cells. J Immunol 174:1922-1931.
Feinberg, M. B., and R. Ahmed. 2012. Born this way? Understanding the immunological basis of effective HIV control. Nat Immunol 13:632-634.
Gonzalez, S., A. Lopez-Soto, B. Suarez-Alvarez, A. Lopez-Vazquez, and C. Lopez-Larrea. 2008. NKG2D ligands: key targets of the immune response. Trends Immunol 29:397-403.
Gonzalez, S., J. Martinez-Borra, J. C. Torre-Alonso, S. Gonzalez-Roces, J. Sanchez del Rio, A. Rodriguez Perez, C. Brautbar, and C. Lopez-Larrea. 1999. The MICA-A9 triplet repeat polymorphism in the transmembrane region confers additional susceptibility to the development of psoriatic arthritis and is independent of the association of Cw*0602 in psoriasis. Arthritis Rheum 42:1010-1016.
Groh, V., S. Bahram, S. Bauer, A. Herman, M. Beauchamp, and T. Spies. 1996. Cell stress-regulated human major histocompatibility complex class I gene expressed in gastrointestinal epithelium. Proc Natl Acad Sci USA 93:12445-12450.
Groh, V., R. Rhinehart, J. Randolph-Habecker, M. S. Topp, S. R. Riddell, and T. Spies. 2001. Costimulation of CD8alphabeta T cells by NKG2D via engagement by MIC induced on virus-infected cells. Nat Immunol 2:255-260.
Groh, V., J. Wu, C. Yee, and T. Spies. 2002. Tumour-derived soluble MIC ligands impair expression of NKG2D and T-cell activation. Nature 419:734-738.
Hankey, K. G., C. B. Drachenberg, J. C. Papadimitriou, D. K. Klassen, B. Philosophe, S. T. Bartlett, V. Groh, T. Spies, and D. L. Mann. 2002. MIC expression in renal and pancreatic allografts. Transplantation 73:304-306.
Holdenrieder, S., P. Stieber, A. Peterfi, D. Nagel, A. Steinle, and H. R. Salih. 2006. Soluble MICA in malignant diseases. Int J Cancer 118:684-687.
Ishitani, A., and D. E. Geraghty. 1992. Alternative splicing of HLA-G transcripts yields proteins with primary structures resembling both class I and class II antigens. Proc Natl Acad Sci USA 89:3947-3951.
Katsuyama, Y., M. Ota, H. Ando, S. Saito, N. Mizuki, J. Kera, S. Bahram, Y. Nose, and H. Inoko. 1999. Sequencing based typing for genetic polymorphisms in exons, 2, 3 and 4 of the MICA gene. Tissue Antigens 54:178-184.
Kloverpris, H. N., M. Harndahl, A. J. Leslie, J. M. Carlson, N. Ismail, M. van der Stok, K. H. Huang, F. Chen, L. Riddell, D. Steyn, D. Goedhals, C. van Vuuren, J. Frater, B. D. Walker, M. Carrington, T. Ndung'u, S. Buus, and P. Goulder. 2012a. HIV control through a single nucleotide on the HLA-B locus. J Virol
Kloverpris, H. N., A. Stryhn, M. Harndahl, M. van der Stok, R. P. Payne, P. C. Matthews, F. Chen, L. Riddell, B. D. Walker, T. Ndung′u, S. Buus, and P. Goulder. 2012b. HLA-B*57 Micropolymorphism shapes HLA allele-specific epitope immunogenicity, selection pressure, and HIV immune control. J Virol 86:919-929.
Krangel, M. S. 1986. Secretion of HLA-A and -B antigens via an alternative RNA splicing pathway. J Exp Med 163:1173-1190.
Kubin, M., L. Cassiano, J. Chalupny, W. Chin, D. Cosman, W. Fanslow, J. Mullberg, A. M. Rousseau, D. Ulrich, and R. Armitage. 2001. ULBP1, 2, 3: novel MHC class I-related molecules that bind to human cytomegalovirus glycoprotein UL16, activate NK cells. Eur J Immunol 31:1428-1437.
Li, P., D. L. Morris, B. E. Willcox, A. Steinle, T. Spies, and R. K. Strong. 2001. Complex structure of the activating immunoreceptor NKG2D and its MHC class I-like ligand MICA. Nat Immunol 2:443-451.
Li, Z., V. Groh, R. K. Strong, and T. Spies. 2000. A single amino acid substitution causes loss of expression of a MICA allele. Immunogenetics 51:246-248.
Livak, K. J., and T. D. Schmittgen. 2001. Analysis of relative gene expression data using real-time quantitative PCR and the 2(-Delta Delta C(T)) Method. Methods 25:402-408.
Mondelli, M. U. 2012. NKG2D and its ligands: key to immunotherapy of liver cancer? J Hepatol 56:308-310.
Nilsen, T. W., and B. R. Graveley. 2010. Expansion of the eukaryotic proteome by alternative splicing. Nature 463:457-463.
Obuchi, N., M. Takahashi, T. Nouchi, M. Satoh, T. Arimura, K. Ueda, J. Akai, M. Ota, T. Naruse, H. Inoko, F. Numano, and A. Kimura. 2001. Identification of MICA alleles with a long Leu-repeat in the transmembrane region and no cytoplasmic tail due to a frameshift-deletion in exon 4. Tissue Antigens 57:520-535.
Ogasawara, K., J. A. Hamerman, L. R. Ehrlich, H. Bour-Jordan, P. Santamaria, J. A. Bluestone, and L. L. Lanier. 2004. NKG2D blockade prevents autoimmune diabetes in NOD mice. Immunity 20:757-767.
Ogasawara, K., and L. L. Lanier. 2005. NKG2D in NK and T cell-mediated immunity. J Clin Immunol 25:534-540.
Paul, P., F. A. Cabestre, E. C. Ibrahim, S. Lefebvre, I. Khalil-Daher, G. Vazeux, R. M. Quiles, F. Bermond, J. Dausset, and E. D. Carosella. 2000. Identification of HLA-G7 as a new splice variant of the HLA-G mRNA and expression of soluble HLA-G5, -G6, and -G7 transcripts in human transfected cells. Hum Immuno161:1138-1149.
Raulet, D. H. 2003. Roles of the NKG2D immunoreceptor and its ligands. Nat Rev Immunol 3:781-790.
Riegert, P., V. Wanner, and S. Bahram. 1998. Genomics, isoforms, expression, and phylogeny of the MHC class I-related MR1 gene. J Immunol 161:4066-4077.
Romphruk, A. V., A. Romphruk, C. Choonhakarn, C. Puapairoj, H. Inoko, and C. Leelayuwat. 2004.
Major histocompatibility complex class I chain-related gene A in Thai psoriasis patients: MICA association as a part of human leukocyte antigen-B-Cw haplotypes. Tissue Antigens 63:547-554.
Salih, H. R., S. Holdenrieder, and A. Steinle. 2008. Soluble NKG2D ligands: prevalence, release, and functional impact. Front Biosci 13:3448-3456.
Salih, H. R., H. G. Rammensee, and A. Steinle. 2002. Cutting edge: down-regulation of MICA on human tumors by proteolytic shedding. J Immunol 169:4098-4102.
Saposnik, B., E. Lesteven, A. Lokajczyk, C. T. Esmon, M. Aiach, and S. Gandrille. 2008. Alternative mRNA is favored by the A3 haplotype of the EPCR gene PROCR and generates a novel soluble form of EPCR in plasma. Blood 111:3442-3451.
Steinle, A., P. Li, D. L. Morris, V. Groh, L. L. Lanier, R. K. Strong, and T. Spies. 2001. Interactions of human NKG2D with its ligands MICA, MICB, and homologs of the mouse RAE-1 protein family. Immunogenetics 53:279-287.
Stephens, H. A. 2001. MICA and MICB genes: can the enigma of their polymorphism be resolved? Trends Immunol 22:378-385.
Stern-Ginossar, N., and O. Mandelboim. 2009. An integrated view of the regulation of NKG2D ligands. Immunology 128:1-6.
Strong, R. K. 2002. Asymmetric ligand recognition by the activating natural killer cell receptor NKG2D, a symmetric homodimer. Mol Immuno138:1029-1037.
Suarez-Alvarez, B., R. Alonso-Arias, C. Bravo-Mendoza, A. Lopez-Vazquez, T. Ortega, J. M. Baltar, E.
Coto, F. Ortega, and C. Lopez-Larrea. 2009a. Identification of epitopes and immunodominant regions on the MICA protein defined by alloantibodies from kidney transplant patients. Transplantation 88:S68-77.
Suarez-Alvarez, B., A. Lopez-Vazquez, J. M. Baltar, F. Ortega, and C. Lopez-Larrea. 2009b. Potential role of NKG2D and its ligands in organ transplantation: new target for immunointervention. Am J Transplant 9:251-257.
Sumitran-Holgersson, S., H. E. Wilczek, J. Holgersson, and K. Soderstrom. 2002. Identification of the nonclassical HLA molecules, mica, as targets for humoral immunity associated with irreversible rejection of kidney allografts. Transplantation 74:268-277.
Sun, D., X. Wang, H. Zhang, L. Deng, and Y. Zhang. 2011. MMP9 mediates MICA shedding in human osteosarcomas. Cell Biol Int 35:569-574.
Sutherland, C. L., N. J. Chalupny, K. Schooley, T. VandenBos, M. Kubin, and D. Cosman. 2002. UL16-binding proteins, novel MHC class I-related proteins, bind to NKG2D and activate multiple signaling pathways in primary NK cells. J Immunol 168:671-679.
Terasaki, P. I., M. Ozawa, and R. Castro. 2007. Four-year follow-up of a prospective trial of HLA and MICA antibodies on kidney graft survival. Am J Transplant 7:408-415.
Tonnerre, P., N. Gerard, M. Chatelais, and B. Charreau. 2010. MICA gene polymorphism in kidney allografts and possible impact of functionally relevant variants. Transplant Proc 42:4318-4321.
Waldhauer, I., D. Goehlsdorf, F. Gieseke, T. Weinschenk, M. Wittenbrink, A. Ludwig, S. Stevanovic, H. G. Rammensee, and A. Steinle. 2008. Tumor-associated MICA is shed by ADAM proteases. Cancer Res 68:6368-6376.
Wang, X., A. D. Lundgren, P. Singh, D. R. Goodlett, S. R. Plymate, and J. D. Wu. 2009. An six-amino acid motif in the alpha3 domain of MICA is the cancer therapeutic target to inhibit shedding. Biochem Biophys Res Commun 387:476-481.
Wang, Z., and C. B. Burge. 2008. Splicing regulation: from a parts list of regulatory elements to an integrated splicing code. RNA 14:802-813.
Xu, X., W. Xia, L. Tian, Y. Chen, H. Ding, Y. Shao, J. Deng, J. Wang, Y. Huang, S. Santoso, Y. Fu, and X. Ye. 2012. Distribution of MICA haplotypes in a Chinese Han population. Hum Immunol 73:75-79.
Zou, Y., and P. Stastny. 2002. Alternatively spliced forms of MICA and MICB lacking exon 3 in a human cell line and evidence of presence of similar RNA in human peripheral blood mononuclear cells. Immunogenetics 54:671-674.
Zwirner, N. W., K. Dole, and P. Stastny. 1999. Differential surface expression of MICA by endothelial cells, fibroblasts, keratinocytes, and monocytes. Hum Immunol 60:323-330.
Zwirner, N. W., M. A. Fernandez-Vina, and P. Stastny. 1998. MICA, a new polymorphic HLA-related antigen, is expressed mainly by keratinocytes, endothelial cells, and monocytes. Immunogenetics 47:139-148.
Zwirner, N. W., C. Y. Marcos, F. Mirbaha, Y. Zou, and P. Stastny. 2000. Identification of MICA as a new polymorphic alloantigen recognized by antibodies in sera of organ transplant recipients. Hum immunol 61:917-924.

1-2. (canceled)

3. A polypeptide which comprises
i) a sequence having at least 80%, 85%, 95%, or 98% identity with, or is 100% identical to, a sequence selected from the group consisting of SEQ ID NO:1 (MICA-A), SEQ ID NO:2 (MICA-B1), SEQ ID NO:3 (MICA-B2); SEQ ID NO:4 (MICA-C) and SEQ ID NO:5 (MICA-D),
ii) an amino acid sequence having at least 80% identity with a sequence ranging from an amino acid residue at position 86 to an amino acid residue at position 189 in SEQ ID NO:1,
iii) an amino acid sequence having at least 80% identity with a sequence ranging from an amino acid residue at position 86 to an amino acid residue at position 174 in SEQ ID NO:2,
iv) an amino acid sequence having at least 80% identity with a sequence ranging from an amino acid residue at position 86 to an amino acid residue at position 175 in SEQ ID NO:4,
v) a fragment or function conservative variant of i), ii), iii) or iv), or
vi) an isolated form of i), ii), iii) or iv).

4-6. (canceled)

7. The polypeptide of claim 3, wherein said fragment
i) comprises all or a portion of an extracellular domain of a polypeptide having an amino acid sequence set forth as SEQ ID NO:2 (MICA-B1), SEQ ID NO:3 (MICA-B2) or SEQ ID NO:5 (MICA-D),
ii) is at least 80, 81, 82, 83, 84, 84, 85, 86, 87, 88, 89, 90, 91, 92, 93, 94, 95, 96, 97, 98, 99% homologous to said extracellular domain, or
is a soluble form of a polypeptide having an amino acid sequence set forth as SEQ ID NO:2 (MICA-B1), SEQ ID NO:3 (MICA-B2) or SEQ ID NO:5 (MICA-D).

8-9. (canceled)

10. The polypeptide of claim 3, wherein said fragment comprises
an amino acid sequence ranging from an amino acid residue at position 86 to an amino acid residue at position 189 in SEQ ID NO:1,
an amino acid sequence ranging from an amino acid residue at position 86 to an amino acid residue at position 174 in SEQ ID NO:2, or
an amino acid sequence ranging from an amino acid residue at position 86 to an amino acid residue at position 175 in SEQ ID NO:4.

11-12. (canceled)

13. A fusion protein comprising a polypeptide according to claim 3 or a fragment thereof fused to a heterologous polypeptide.

14. The fusion protein of claim 13 which is an immunoadhesin.

15. The fusion protein of claim 14 wherein the immunoadhesin comprises
i) a polypeptide with a sequence set forth as SEQ ID NO:1 (MICA-A) or SEQ ID NO:4 (MICA-C), or
ii) a soluble form of a polypeptide with a sequence set forth as SEQ ID NO:2 (MICA-B1), SEQ ID NO:3 (MICA-B2) or SEQ ID NO:5 (MICA-D).

16. (canceled)

17. A nucleic acid molecule that encodes a polypeptide according to claim 3, or a fusion protein according to claim 13, or a nucleic acid that is complementary to said nucleic acid molecule.

18. The nucleic acid molecule of claim 17, wherein said nucleic acid molecule comprises a nucleotide sequence which is at least 80%, 85%, 95%, or 98% identical, or is 100% identical, to the nucleotide sequence of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 or SEQ ID NO:15.

19-20. (canceled)

21. The nucleic acid molecule of claim 17, wherein said nucleic acid that is complementary is complementary to the nucleotide sequence of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15.

22. The nucleic acid molecule of claim 21 which comprises a region of a nucleotide sequence that hybridizes under stringent conditions to at least about 12, 25, 50, 75, 100, 125, 150, 175, 200, 250, 300, 350 or 400 consecutive nucleotides of a sense or anti-sense sequence of SEQ ID NO:6 (MICA-A), SEQ ID NO:7 (MICA-B1), SEQ ID NO:8 (MICA-B2), SEQ ID NO:9 (MICA-C), SEQ ID NO:10 (MICA-D), SEQ ID NO:11, SEQ ID NO:12, SEQ ID NO:13, SEQ ID NO:14 or SEQ ID NO:15.

23. The nucleic acid molecule of claim 22 which comprises or is a sequence selected from the group consisting of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, and SEQ ID NO:25.

24. An oligonucleotide primer pair selected from the group consisting of
SEQ ID NO:16 and SEQ ID NO:17 for amplifying a nucleic acid molecule with a sequence set forth as SEQ ID NO:6 (MICA-A),
SEQ ID NO:18 and SEQ ID NO:19 for amplifying a nucleic acid molecule with a sequence set forth as SEQ ID NO:7 (MICA-B1),
SEQ ID NO:20 and SEQ ID NO:21 for amplifying a nucleic acid molecule with a sequence set forth as SEQ ID NO:8 (MICA-B2),
SEQ ID NO:22 and SEQ ID NO:23 for amplifying a nucleic acid molecule with a sequence set forth as SEQ ID NO:9 (MICA-C)
SEQ ID NO:24 and SEQ ID NO:25 for amplifying a nucleic acid molecule with a sequence set forth as SEQ ID NO:10 (MICA-D).

25-28. (canceled)

29. The nucleic acid molecule of claim 22, wherein said nucleic acid molecule is at least 100, 200, 300, 400, or 500 contiguous nucleotides in length.

30. The nucleic acid molecule of claim 18, wherein a nucleotide sequence of said nucleic acid molecule differs from a nucleotide sequence selected from the group consisting of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15 due to degeneracy of the genetic code and thus encodes a protein that is the same as that encoded by the nucleotide sequence of SEQ ID NO:6 (MICA-A), SEQ ID NO:7 (MICA-B1), SEQ ID NO:8 (MICA-B2), SEQ ID NO:9 (MICA-C) or SEQ ID NO:10 (MICA-D) SEQ ID NO:11, SEQ ID NO:12, SEQ ID NO:13, SEQ ID NO:14 and SEQ ID NO:15.

31. A recombinant expression vector containing a nucleic acid encoding
a polypeptide according to claim 3 or a fragment thereof, or
a fusion protein according to claim 13.

32. A transformed host cell comprising a recombinant expression vector of claim 31.

33. The host cell of claim 32 which is a prokaryotic or eukaryotic cell.

34. The host cell of claim 32 which is a mammalian cell.

35. A method of producing a polypeptide comprising the steps of: (i) culturing the transformed host cell of claim 32 under conditions suitable to allow expression of said polypeptide; and (ii) recovering the expressed polypeptide.

36. An antibody or aptamer specific for
a polypeptide according to claim 3, or
a fusion protein according to claim 13.

37. The antibody of claim 36 which is selected from the group consisting of monoclonal antibodies, antibody fragments that comprise an antigen binding domain, single domain antibodies, TandAbs dimer, Fv, scFv, dsFv, ds-scFv, Fd, linear antibodies, minibodies, diabodies, bispecific antibody fragments, bibody, tribody; sc-diabody; kappa(lamda) bodies; (dual variable domain antibody, bispecific format (DVD-Ig); small immunoprotein (SIP); small modular immunopharmaceutical scFv-Fc dimer (SMIP); Dual Affinity ReTargeting ds-stabilized diabody (DART); and small antibody mimetics comprising one or more CDRs.

38. The antibody of claim 36 which is a monoclonal antibody.

39. The antibody of claim 36 which is a chimeric antibody, a humanized antibody, or a human antibody.

40. The antibody or aptamer of claim 36 which is specific for
an amino acid sequence ranging from an amino acid residue at position 86 to an amino acid residue at position 189 in SEQ ID NO:1,
an amino acid sequence ranging from an amino acid residue at position 86 to an amino acid residue at position 174 in SEQ ID NO:2, or
an amino acid sequence ranging from an amino acid residue at position 86 to an amino acid residue at position 175 in SEQ ID NO:4.

41-46. (canceled)

47. An immunoconjugate comprising an antibody conjugated to a polypeptide according to claim 3, or a fusion protein according claim 13.

48. The immunoconjugate of claim 47 wherein the antibody is directed against one antigen that is a part of a cell, or against a microorganism selected from the groups consisting of a bacterium, a fungus, a protozoan, and a virus.

49. The immunoconjugate of claim 47 wherein the antibody is directed against a cancer antigen.

50. A pharmaceutical composition comprising
a polypeptide according to claim 3,
a fusion protein according to claim 13,
an antibody according to claim 36 or
an immunoconjugate according to claim 47.

51. A kit comprising (1) a first antibody according to claim 36 and, optionally, (2) a second, different antibody which binds to either the polypeptide according to claim 3 or the first antibody, wherein the second, different antibody is conjugated to a detectable agent.

52. A kit comprising (1) an oligonucleotide which hybridizes to a nucleic acid sequence encoding for a polypeptide according to claim 3 or (2) a pair of primers useful for amplifying a nucleic acid molecule encoding a polypeptide according to claim 3.

53. The kit of claim 52 which comprises
oligonucleotide primers SEQ ID NO:16 and SEQ ID NO:17 for amplifying a nucleic acid molecule with a sequence as set forth in SEQ ID NO:6 (MICA-A),
oligonucleotide primers SEQ ID NO:18 and SEQ ID NO:19 for amplifying a nucleic acid molecule with a sequence as set forth in SEQ ID NO:7 (MICA-B1).
oligonucleotide primers SEQ ID NO:20 and SEQ ID NO:21 for amplifying a nucleic acid molecule with a sequence as set forth in SEQ ID NO:8 (MICA-B2)
oligonucleotide primers SEQ ID NO:22 and SEQ ID NO:23 for amplifying a nucleic acid molecule with a sequence as set forth in SEQ ID NO:9 (MICA-C)
oligonucleotide primers SEQ ID NO:24 and SEQ ID NO:25 for amplifying a nucleic acid molecule with a sequence as set forth in SEQ ID NO:10 (MICA-D).

54-57. (canceled)
